(19) United States
(12) Patent Application Publication
(10) Pub. No.: US 2011/0076304 A1
(43) Pub. Date: Mar. 31, 2011

(54) ADAPTER MOLECULE FOR THE DELIVERY OF ADENOVIRUS VECTORS

(75) Inventors: Alexander Pereboev, Birmingham, AL (US); George Tsuladze, Birmingham, AL (US); Igor Bogdashin, Birmingham, AL (US); Itziar Echeverria Beistegui, Pamplona-Navarra (ES); Juan José Lasarte Sagastibelza, Pamplona-Navarra (ES); Jesùs Maria Prieto Valtueña, Pamplona-Navarra (ES); Pablo Sarobe Ugarriza, Pamplona-Navarra (ES)

(73) Assignees: PROYECTO DE BIOMEDICINA CIMA, S.L., Zizur Mayor - Navarra (ES); THE UAB RESEARCH FOUNDATION, Birmingham, AL (US)

(21) Appl. No.: 12/993,803

(22) PCT Filed: May 19, 2009
(86) PCT No.: PCT/EP2009/056053

§ 371 Date: Nov. 19, 2010

Related U.S. Application Data
(60) Provisional application No. 61/055,332, filed on May 22, 2008.

(51) Int. Cl. A61K 39/235; C12N 7/00; A61P 37/04

(52) U.S. Cl. 424/196.11; 435/235.1

(57) ABSTRACT

The invention relates to an adapter protein comprising a coxackievirus and adenovirus receptor (CAR) region and a human CD40 ligand and to the uses thereof for promoting adenoviral transduction of dendritic cells while at the same time promoting maturation of the DCs. The invention also relates to pharmaceutical compositions comprising said adapter protein and an adenovirus encoding an antigen and the uses thereof in a method for eliciting an immune response against the antigen encoded in said adenovirus as well as to antigen-loaded dendritic cells obtained the adaptor protein and an adenovirus and to the uses thereof in a method of eliciting an immune response against the antigen encoded in the adenovirus.

### CROSS-REFERENCE TO PRIORITY APPLICATIONS

This application claims priority to U.S. Provisional Application No. 61/055,332, filed May 22, 2008, which is incorporated herein by reference in its entirety.

### STATEMENT REGARDING FEDERALLY FUNDED RESEARCH

This invention was made with government funding under Grant No. 5 U54 AI057157-04 from the National Institutes of Health. The government has certain rights in this invention.

### BACKGROUND OF THE INVENTION

The hepatitis C virus (HCV) infection is characterized by its high tendency towards chronicity, which in some cases can progress to cirrhosis and eventually to hepatocarcinoma. The prevalence of this infection has been estimated at 1-2%, which added to the low efficacy of currently existing therapies for treating the chronic phase of the infection makes it very important to develop a vaccine. The importance of the immune response in HCV infection has been emphasized by means of studies which have demonstrated that those individuals who manage to eliminate the viral infection have a potent and multi-specific cellular immune response, whereas chronically infected patients hardly present response, and it is focused on very few regions of the viral antigens.

Among the different strategies for generating an immune response towards HCV, dendritic cell (DC)-based vaccines has become increasingly popular during the past several years. Dendritic cells (DCs) are a heterogeneous cell population which is characterized by being professional antigen-presenting cells (APCs). In the absence of infection or inflammation, DCs are in an immature or rest state, whereas after an infection or during an inflammatory process, they undergo an activation process known as maturation. In this process, DCs acquire the capacity to migrate to lymphoid organs and present antigens to T lymphocytes for their correct activation.

Approaches to genetically modify DC include gene delivery utilizing liposomes (Copland et al., 2003, Vaccine, 21:883-890) and viral vectors (Jenne et al., 2001, Trends Immunol., 22:102-107).

Adenovirus (Ad)-mediated gene delivery seems attractive due to outstanding efficiency in vitro and in vivo, large payload capacity of Ad vectors, and their ability to infect both dividing and quiescent cells. However, application of Ad vectors for DC modification is hindered by the lack of expression of the primary Ad receptor, CAR, on DC of human and murine origin.

Different strategies have been developed in order to improve the efficiency of DC infection by Ad vectors. For instance, Kita-Furuyama et al. (Clin. Exp. Immunol., 2003, 131:234-240) have described the use of higher viral doses to achieve Ad-mediated gene transfer to DC.

Alternatively, different systems of CAR-independent Ad-mediated transfer of DC have been reported. Some methods rely on modified Ad vectors wherein the fiber proteins have been modified so as to increase the adenoviral tropism towards dendritic cells. For instance, WO0393455 describes modified Ad vectors carrying fiber proteins of the adenoviral B subgroup. US2008124360 describes modified Ad vectors carrying fiber proteins of adenoviral D vectors. US2008003236 describes modified Ad vectors wherein the CAR binding regions and the RGD regions in the fiber protein and the fiber protein has been replaced by the shaft of a type C adenovirus. However, the systems based on recombinant adenoviruses showing altered tropism may be faced with problems due to broad tropism of the modified fiber proteins, resulting in a low cell specificity and making then unsuitable for in vivo approaches. Moreover, these systems require constructing modified adenoviral vectors which is usually time-consuming.

### SUMMARY OF THE INVENTION

In a first aspect, the invention relates to a polypeptide comprising
(i) a domain of coxsackievirus and adenovirus receptor (CAR) capable of binding to an adenoviral fiber protein or a functional variant thereof,
(ii) a trimerization motif and
(iii) a human CD40 ligand.

In further aspects, the invention relates to a nucleic acid encoding a polypeptide as defined above, to a vector comprising said nucleic acid, to a host cell comprising a polypeptide as defined above, a nucleic acid as defined above or a vector as defined above and to a method of making a polypeptide as defined above, and more particularly a polypeptide comprising an ectodomain of CAR, a trimerization motif, and a fragment of a human CD40 ligand, the method comprising:
(a) culturing a host cell as defined above under conditions that allow production of the polypeptide; and
(b) isolating the polypeptide.

In further aspects, the invention relates to a composition or to a complex comprising:
(a) a polypeptide as defined above and
(b) an adenovirus encoding an antigen.
   as well as to a pharmaceutical composition comprising a composition or complex of the invention and a pharmaceutically acceptable carrier.

In another aspect, the invention relates to a method of eliciting an immune response against an antigen in a subject comprising the steps of administering to the subject a complex comprising:
(a) a polypeptide of the invention and
(b) an adenovirus encoding an antigen.

In another aspect, the invention relates to a method of obtaining an antigen-loaded CD40-positive antigen-presenting cell, comprising the steps of (i) contacting a CD40-positive antigen-presenting cell with a polypeptide of
the invention and an adenovirus encoding an antigen, wherein said contacting can be carried out by separately adding the polypeptide and the adenovirus or by adding a preformed polypeptide-adenovirus complex,
(ii) maintaining the mixture obtained in step (i) under conditions adequate for the formation of a ternary complex between said polypeptide, said adenovirus and said cell and
(iii) maintaining the cells under conditions adequate for internalization, processing and presentation of one or more peptides derived from the antigen.

In further aspects, the invention relates to an antigen-loaded CD40-positive antigen-presenting cell obtained by the method defined above as well as to a method of eliciting an immune response in a subject comprising administration to a subject of the antigen presenting cell of the invention.

### BRIEF DESCRIPTION OF THE DRAWINGS

FIG. 1. The use of CFm40L increases the efficacy of transduction of DCs with adenovirus. Graph showing the efficiency of transduction of DCs with a recombinant adenovirus encoding the green fluorescent protein in the presence or absence of the adapter molecule CFm40L. The results are represented as the percentage of transduced cells (GFP+) for each of the amounts of virus used.

FIG. 2. The transduction of DCs with AdNS3 in the presence of CFm40L induces their in vitro maturation: expression of surface markers. FACS analysis of CD54, CD80, CD86, I-Ab surface marker expression in DCs incubated in the presence or absence of CFm40L. The numbers indicate the mean fluorescence value (in arbitrary units) for each of the histograms.

FIG. 3. The transduction of DCs with AdNS3 in the presence of CFm40L induces their in vitro maturation: production of cytokines ELISA determination of IL-12, IL-10 and IL-6 produced by DCs incubated in the presence or absence of CFm40L.

FIG. 4. The maturation of DCs induced by CFm40L is accompanied by the expression of Notch ligands associated to the induction of Th1 responses. Expression fold increase of DLL4, Jagged 1 and Jagged 2 in DCs in response to CFm40L. The results are represented standardized with actin and shown as degree of induction relative to untreated DCs.

FIG. 5. The transduction of DCs with AdNS3 in the presence of CFm40L increases their in vitro stimulatory capacity. [³H] thymidine incorporation (A), IFN-gamma production (B) and IL-4 production (C) in lymphocytes cultured in the presence of allogenic DCs transduced with adenoviral vectors in the presence of absence of CFm40L. Number of HCV NS3-specific lymphocytes producing IFN-γ as measured by ELISPOT when cultured with syngenic DCs transduced with adenoviral vectors in the presence of absence of CFm40L (D). Results are given in IFN-gamma Spot Forming Cells (SFC).

FIG. 6. The immunization with DCs transduced with AdNS3 together with CFm40L induces more potent responses than with DCs and AdNS3 alone. (A) Number of splenocytes producing IFN-γ isolated from mice injected with DCs previously transduced with AdNS3 in the presence or absence of CFm40L in response to stimulation with NS3 CD8 epitopes 1038-1047, 1073-1081, 1406-1415 or recombinant NS3 protein. (B) ELISPOT-determination of the number of splenocytes producing IFN-γ as shown in (A) the NS3 peptides 1367, 1427 and 1447, described in Zabaleta et al, Mol Ther. 2008, 16:210-7)

FIG. 7. CFh40L enhances Ad transduction of human CD40-expressing cells. Luciferase activity of CD40-expressing 293 cells transduced with Ad encoding luciferase in the absence or in the presence of different concentrations of CFh40L. Results are given as relative lights units (RLU).

FIG. 8. The use of CFh40L increases the efficacy of transduction of DCs with adenoviruses. Percentage of DCs transduced with AdGFP at 30 or 300 moi in the presence or absence of the adapter CFh40L. The results are represented as the percentage of transduced cells (GFP+) for each of the amounts of virus used.

FIG. 9. The transduction of human DCs with AdNS3 in the presence of CFh40L induces their in vitro maturation: expression of surface markers FACS analysis of the CD54, CD80, CD86 and HLA-DR surface marker expression in human DCs left untreated or treated with the adapter CFh40L, with poly(I:C) or with a cocktail containing TNF-α, Ampligen and IFN-α. The results show the values of each of the markers as the Mean Fluorescence Index (MFI).

FIG. 10. The transduction of human DCs with AdNS3 in the presence of CFh40L induces their in vitro maturation: production of IL-12. IL-12 levels in supernatants from cultured human DCs left untreated, transduced with AdNS3, transduced with AdNS3 in the presence of the adapter CFh40L, transduced with AdNS3 in the presence of poly(I:C) or transduced with AsNS3 in the presence of a cocktail containing TNF-α, Ampligen and IFN-α

FIG. 11. The transduction of human DCs with AdNS3 in the presence of CFh40L induces their in vitro maturation: stimulation of allogeneic T cells. [³H] thymidine incorporation in DCs treated with AdNS3, AdNS3+CFh40L or AdNS3+TNF-α+Ampligen+IFN-α or AdNS3+CFh40L.

FIG. 12. The transduction with AdNS3 of DCs derived from monocytes obtained from patients with chronic hepatitis C virus infection, in the presence of CFh40L, induces a cellular activation similar to that found in DCs obtained from healthy HCV-seronegative individuals. Expression of CD80, CD86, HLA-DR and CD54 surface markers by flow cytometry (A), production of IL-12 in the culture supernatants (B) and capacity to stimulate allogeneic T lymphocytes (C) in response to the treatment with AdNS3 in the presence of CFh40L in DCs obtained from monocytes from healthy subjects or HCV-infected patients.

### DETAILED DESCRIPTION OF THE INVENTION

The authors of the present invention have observed that, surprisingly, a bifunctional adapter comprising the hCD40L, a trimerization motif and the human CAR ectodomain allows adenoviral vectors to efficiently transduce human DCs while at the same time promotes activation of said DCs into cells capable of presenting an antigen encoded by said adenovirus.

Adapter Polypeptide of the Invention

Thus, in a first aspect, the invention relates to a polypeptide comprising:
(i) a domain of coxsackievirus and adenovirus receptor (CAR) capable of binding to an adenoviral fiber protein or a functional variant thereof,
(ii) a trimerization motif and
(iii) a human CD40 ligand.

As used herein, the term “coxsackievirus and adenovirus receptor” or “CAR” relates to a 46 kDa transmembrane protein that is a member of the immunoglobulin superfamily which acts as primary receptor for Ad subgroups A (e.g. Ad12), C (e.g. Ad2 and Ad5), D (e.g. Ad8, Ad9, Ad10, Ad13, Ad15, Ad17, Ad19, Ad20, Ad22, Ad30, Ad32, Ad33, Ad36-39 and 42-49), E and F (Ad40 and Ad41) as well as for Coxsackie B viruses.

Preferred CAR proteins for use in the present invention include, without limitation, human CAR, rat CAR and mouse CAR.

Human CAR (UniProt Accession number P78310 and depicted in SEQ ID NO:1) is a 365 amino acids polypeptide wherein amino acids 1-19 form a signal sequence and amino acids 20-365 form the mature CAR protein. The soluble region of CAR ectodomain is formed by amino acids 20-237, wherein amino acids 20-134 form the Ig-like C2-type 1 domain and amino acids 141-228 form the Ig-like C2-type 2.

Rat CAR (UniProt Accession number Q9R066 and depicted in SEQ ID NO:2) is a 365 amino acids polypeptide wherein amino acids 1-19 form a signal sequence and amino acids 20-365 form the mature CAR protein. The soluble region of CAR ectodomain is formed by amino acids 20-238, wherein amino acids 20-136 form the Ig-like C2-type 1 domain and amino acids 141-228 form the Ig-like C2-type 2.

Mouse CAR (UniProt Accession number P97792 and depicted in SEQ ID NO:3) is a 365 amino acids polypeptide wherein amino acids 1-19 form a signal sequence and amino acids 20-365 form the mature CAR protein. The soluble region of CAR ectodomain is formed by amino acids 20-237, wherein amino acids 20-136 form the Ig-like C2-type 1 domain and amino acids 141-228 form the Ig-like C2-type 2.

The term “domain of CAR capable of binding to an adenoviral fiber protein” refers to any region from CAR, preferably, from the extracellular domain of CAR which, when expressed in a target cell, allows infection of said cell by an adenovirus. The domain may comprise the complete extracellular region (amino acids 20-237 of the human CAR, amino acids 20-237 of the mouse CAR or amino acids 20-238 of the rat CAR), the Ig-like Cl domain (amino acids 20-134 of the human CAR, amino acids 20-136 of the rat CAR or amino acids 20-134 of the mouse CAR), the Ig-like C2 domain (amino acids 141-228 of the human CAR, amino acids 141-228 of the rat CAR and amino acids 141-228 of the mouse CAR), a region comprising both the Ig-like Cl and the Ig-like C2 domains or any region which is capable of binding to the adenoviral fiber protein with sufficient specificity so as to ensure efficient infection of cells expressing said receptor. By way of an example, the determination for the binding capacity of a CAR domain to the adenoviral fiber protein can be carried out by surface plasmon resonance as described in Kirby et al. (J. Virol., 2000, 74:2804-2813). Suitable domains for use in the adapter molecules of the present invention include those having a binding constant of at least 10⁻⁷ M, preferably at least 10-8 M, more preferably of at least 9×10⁻⁹ M, at least 8×10⁻⁹ M, at least 7×10⁻⁹ M, at least 6×10⁻⁹ M, at least 5×10⁻⁹ M, at least 4×10⁻⁹ M, at least 3×10⁻⁹ M, at least 2×10⁻⁹ M, at least 10^{]}M, at least 9×10⁻¹⁰ M, at least 8×10⁻¹⁰ M, at least 7×10⁻¹⁰ M, at least 6×10⁻¹⁰ M, at least 5×10⁻¹⁰ M, at least 4×10⁻¹ M, at least 3×10⁻¹⁰ M, at least 2×10⁻¹⁰ M, at least 10⁻¹⁰ M.

The term “functional variant”, as used herein, relates to any polypeptide derived from CAR by insertion, deletion or substitution of one or more residues and which maintains substantially the ability to interact with the adenoviral fiber protein as determined above. Suitable functional variants are those showing a degree of identity with respect to the CAR domain of about greater than 25% amino acid sequence identity, such as 25%, 40%, 60%, 70%, 80%, 90% or 95%. The degree of identity between two polypeptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)]. BLAST and BLAST 2.0 are used, with the parameters described herein, to determine percent sequence identity. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always 0) and N (penalty score for mismatching residues; always 0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. Suitable values of the BLASTP parameters are, without limitation, the default values of a wordlength of 3, and expectation (E) of 10, and the BLOSUM62 scoring matrix (see Henikoff and Henikoff, Proc. Natl. Acad. Sd. USA, 1989, 89:10915) alignments (B) of 50, expectation (E) of 10, M=5, N=−4, and a comparison of both strands.

In a preferred embodiment, the CAR domain is an ectodomain of human CAR. In a still more preferred embodiment, the CAR domain comprises amino acids 1-263 of SEQ ID NO:1. In a still more preferred embodiment, the CAR domain consists of amino acids 1-263 of SEQ ID NO:1.

The second component of the polypeptide of the invention is a trimerization motif As used herein, the term “trimerization motif” or “trimerizing motif” relates to an amino acid sequence that comprises the functionality that can associate with two other amino acid sequences to form trimers. A trimerizing motif or domain can associate with other trimerizing domains of identical amino acid sequence (a homotrimer), or with trimerizing domains of different amino acid sequence (a heterotrimer). Such an interaction may be caused by covalent bonds between the components of the trimerizing domains as well as by hydrogen bond forces, hydrophobic forces, van der Waals forces and salt bridges.

Suitable trimerizing domains are, without limitation, the tetranectin trimerizing structural element (TTSE) as described in U.S. Patent Application Publication No. 2007/0154901, the trimerization motif present in the C-terminal region of the acetyl choline receptor CoIQ chain as described in WO06076024, the trimerization motif of the GCN4 leucine zipper (Harbury et al. 1993 Science 262:1401-1407), the trimerization motif from the lung surfactant protein (Hoppe et al. 1994, FEBS Lett 344:191-195), the trimerization motif of collagen (McAlinden et al. 2003 J Biol Chem 278:42200-42207), the trimierization domain of collagen XVIII NCl domain, the trimerization motif of TNF, the E. coli skp trimerization motif, the trimerization motif of the adenovirus fiber protein, the trimerization motif of human matrilin as described by Dames S A. Et al (Nat Struct Biol., 1998; 5: 687-91), the trimerization motif of NEMO as described by Veron, M. et al. (J Biol Chem, 2004, 279:27861-27869), the tenascin trimerization motif as described in WO09000538A, the colied coil region of the macrophage scavenger receptor as described in by Frank et al (J. Biol. Chem., 2000, 275: 11672-11677) and the phage T4 fibritin ‘foldon’ (Miroshnikov et al. 1998 Protein Eng 11:329-414).

In a preferred embodiment, the trimerization motif is the phage T4 fibritin ‘foldon’ as defined by SEQ ID NO:4 (GYIPEAPRDGQAYVRKDGEWVLLSTF). This sequence adopts a beta-propeller conformation, and can fold and trimerize in an autonomous way (Tao et al. 1997 Structure 5:789-798). In another preferred embodiment, the trimerization motif is the neck region peptide (NRP) from the human lung surfactant protein D as defined by SEQ ID NO:5 (PDVASLRQQVEALQGQVQHLQAAFSQYKKVELFPNG)

The third element of the polypeptide of the invention is the human CD40 ligand. As used herein, “CD40Ligand” (CD40L) shall encompass any polypeptide or protein that specifically recognizes and activates the CD40 receptor and activates its biological activity. While the term does not exclude the use of a CD40L containing the transmembrane domain, it is preferred to use a soluble forms of CD40L containing all or part of the extracellular domain.

A human CD40L amino acid sequence is shown in SEQ ID NO:6.

Suitable CD40L fragments for use in the polypeptide of the invention include, without limitation, a truncated CD40L comprising residues 47 to 261 of SEQ ID NO:6, a CD40L fragment comprising amino acid residues 51 through 261 of SEQ ID NO:6; a CD40L fragment comprising amino acid residues 120 through 261 of SEQ ID NO:6; a CD40L fragment comprising amino acid residues 113 through 261 of SEQ ID NO:6; a CD40 fragment comprising amino acid residues 112 through 261 of SEQ ID NO:6; a CD40 fragment comprising amino acid residues 35 through 261 of SEQ ID NO:6; a CD40L fragment comprising amino acid residues 34 through 225 of SEQ ID NO:6; a CD40L fragment comprising amino acid residues 113 through 225 of SEQ ID NO:6; a CD40L fragment comprising amino acid residues 120 through 225 of SEQ ID NO:6. In a preferred embodiment, the fragment of human CD40 ligand comprises amino acids 118 to 231of SEQ ID NO:6.

A human CD40L suitable for use in the present invention include variants of the CD40L obtained by mutations of nucleotide sequences coding for a CD40L polypeptide and which preserve substantially the capacity of binding CD40. Suitable methods for determining whether a CD40L variant maintains the capacity of binding to CD40 include conventional binding assays that may be carried out using any conventional technologies such as surface plasmon resonance as described by Wieckowski S. et al. (Biochemistry, 2007, 46:3482-93) or by binding onto immobilized CD40 as described by Mazzei et al. (J. Biol. Chem., 1995, 270:7025-7028).

A CD40L analog, as referred to herein, is a polypeptide substantially homologous to a sequence of human or murine CD40L but which has an amino acid sequence different from native sequence CD40L polypeptide because of one or a plurality of deletions, insertions or substitutions. Generally, substitutions should be made conservatively; i.e., the most preferred substitute amino acids are those which do not affect the ability of the inventive proteins to bind their receptors in a manner substantially equivalent to that of native CD40L. Suitable functional variants are those showing a degree of identity with respect to human CD40L of about greater than 25% amino acid sequence identity, such as 25% 40%, 60%, 70%, 80%, 90% or 95%. Moreover, the primary amino acid structure of human CD40L or variant thereof may be modified to create CD40L derivatives by forming covalent or aggregative conjugates with other chemical moieties, such as glycosyl groups, lipids, phosphate, acetyl groups and the like, or by creating amino acid sequence mutants. Covalent derivatives of CD40L are prepared by linking particular functional groups to CD40L amino acid side chains or at the N-terminus or C-terminus of a CD40L polypeptide or the extracellular domain thereof. Other derivatives of CD40L within the scope of this invention include covalent or aggregative conjugates of CD40L or its fragments with other proteins or polypeptides, such as by synthesis in recombinant culture as N-terminal or C-terminal fusions. For example, the conjugate may comprise a signal or leader polypeptide sequence at the N-terminal region or C-terminal region of a CD40L polypeptide which co-translationally or post-translationally directs transfer of the conjugate from its site of synthesis to a site inside or outside of the cell membrane or cell wall (e.g. the [alpha]-factor leader of Saccharomyces).

In a preferred embodiment, the adapter molecule of the invention further comprises a tag. The term “tag”, as used herein, relates to any amino acid sequence for which specific binding molecules are available, thus allowing the detection/purification of any polypeptide carrying said tag. The tag is generally placed at the amino- or the carboxyl-terminus of the polypeptide. The presence of such tag allows the adapter molecule to be detected using an antibody against the tag polypeptide. Also, provision of the tag enables the adapter polypeptide to be readily purified by affinity purification using an anti-tag antibody or another type of affinity reagent that binds to the epitope tag.

Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., 1988, Mol. Cell. Biol., 8: 2159-2165); the c-myc tag and the 8F9, 3C7, 6E10, G4, B7 and 9E10 antibodies thereto (Evan et al., 1985, Molecular and Cellular Biology, 5:3610-3616); the Herpes Simplex virus glycoprotein D (gD) tag and its antibody (Paborsky et al., 1990, Protein Engineering, 3:547-553). Other tag polypeptides include the Flag-peptide (Hopp et al., 1988, BioTechnologv, 6:1204-1210); the KT3 epitope peptide [Martin et al., 1993, Science, 255: 192-194); tubulin epitope peptide (Skinner et al., 1991, J. Biol. Chem., 266: 15163-15166); and the T7 gene 10 protein peptide tag (Lutz-Freyermuth et al., 1990, Proc.Natl.Acad.Sci.USA, 87:6393-6397). In a preferred embodiment, the purification tag is a polyhistidine tag. In a still more preferred embodiment, the purification tag is an hexahistidine tag.

The skilled person will appreciate that the different elements of the polypeptide of the invention may be arranged in any order as long as the tridimensional structure of the CD40L and of the CAR region are preserved and maintain the function of interaction with CD40 or interaction with the adenoviral fiber proteins. Thus, suitable arrangements of the adapter polypeptide of the invention include:
CAR domain-trimerization motif-CD40L
CAR domain-CD40L-trimerization motif
Trimerization motif-CAR domain-CD40L
Trimerization motif-CD40L-CAR domain
CD40L-Trimerization motif-CAR domain
CD40L-CAR domain-Trimerization motif

In a preferred embodiment, the adapter protein comprises, in order from the N-terminus, CAR domain capable of binding to an adenoviral fiber protein, the trimerization motif and the CD40L. In a still more preferred embodiment, the adapter protein comprises the following elements in order from the N-terminus: CAR domain capable of binding to an adenoviral fiber protein, a linker region, an hexahistidine tag and the hCD40L.

The different elements of the polypeptide of the invention may be attached directly, i.e. the C-terminus of an element is linked directly to the N-terminal region of the following element. However, it is also possible that the elements are contacted via a linker region.

According to the invention, said linker region sequence acts as a hinge region between the CAR domain and the human CD40L, allowing them to move independently from one another while they maintain the three-dimensional shape of the individual domains. In this sense, a preferred non-natural intermediate amino acid sequence according to the invention would be a hinge region characterized by a structural ductility allowing this movement. In a particular embodiment, said non-natural intermediate amino acid sequence is a non-natural flexible linker In a preferred embodiment, said flexible linker is a flexible linker peptide with a length of 20 amino acids or less. In a more preferred embodiment, the linker peptide comprises 2 amino acids or more selected from the group consisting of glycine, serine, alanine and threonine. In a preferred embodiment of the invention, said flexible linker is a polyglycine linker. Possible examples of linker/spacer sequences include SGGTSGSTSGTGST (SEQ ID NO:7), AGSSTGSSTGPGSTT (SEQ ID NO:8) or GGSGGAP (SEQ ID NO:9) and GGGVEGGG (SEQ ID NO: 10). These sequences have been used for binding designed coiled helixes to other protein domains (Muller, K. M., Arndt, K. M. and Alber, T., Meth. Enzymology, 2000, 328: 261-281). Said linker preferably comprises or consists of the amino acid sequence GGPGS (SEQ ID NO: 11).

The effect of the linker region is providing space between the CAR domain and the human CD40L. It is thus ensured that the secondary structure of CAR is not affected by the presence the hCD40L and vice versa. The spacer preferably has a peptide nature. The linker peptide preferably comprises at least two amino acids, at least three amino acids, at least five amino acids, at least ten amino acids, at least 15 amino acids, at least 20 amino acids, at least 30 amino acids, at least 40 amino acids, at least 50 amino acids, at least 60 amino acids, at least 70 amino acids, at least 80 amino acids, at least 90 amino acids or approximately 100 amino acids.

The linker can be bound to components flanking the two components of the conjugates of the invention by means of covalent bonds and preferably the spacer is essentially non-immunogenic and/or does not comprise any cysteine residue. In a similar manner, the three-dimensional structure of the spacer is preferably linear or substantially linear.

Preferred examples of spacer or linker peptides include those which have been used for binding proteins without substantially deteriorating the function of the bound proteins or at least without substantially deteriorating the function of one of the bound proteins. More preferably, the spacers or linkers have been used for binding proteins comprising structures with coiled helixes.

In a preferred embodiment, the linker is placed at the C-terminus of the CAR, i.e. it acts by linking the CAR domain and the trimerization motif.

Polynucleotides, Gene Constructs, Vectors and Host Cells of the Invention.

In another aspect, the invention relates to a polynucleotide encoding an adapter polypeptide of the invention. A person skilled in the art will understand that the polynucleotides of the invention will only encode the adapter molecule regardless of the relative orientation and regardless of the fact that the components of the adapter molecule are directly connected or separated by a spacer region.

The polynucleotide of the invention may be isolated or may form part of a gene construct. The construct preferably comprises the polynucleotide of the invention located under the operative control of sequences regulating the expression of the polynucleotide of the invention. A person skilled in the art will understand that the polynucleotides of the invention must access the nucleus of a target tissue and there be transcribed and translated to give rise to the biologically active fusion protein.

In principle, any promoter can be used for the gene constructs of the present invention provided that said promoter is compatible with the cells in which the polynucleotide is to be expressed. Thus, promoters suitable for the embodiment of the present invention include, without being necessarily limited to, constitutive promoters such as the derivatives of the genomes of eukaryotic viruses such as the polyoma virus, adenovirus, SV40, CMV, avian sarcoma virus, hepatitis B virus, the promoter of the metallothionein gene, the promoter of the herpes simplex virus thymidine kinase gene, retrovirus LTR regions, the promoter of the immunoglobulin gene, the promoter of the actin gene, the promoter of the EF-lalpha gene as well as inducible promoters in which the expression of the protein depends on the addition of a molecule or an exogenous signal, such as the tetracycline system, the NFKB/UV light system, the Cre/Lox system and the promoter of heat shock genes, the regulatable promoters of RNA polymerase II described in WO/2006/135436 as well as tissue-specific promoters. In a preferred embodiment, the gene constructs of the invention contain the expression-enhancing regions present in promoter regions of predominantly hepatic expression genes such as human serum albumin genes, prothrombin genes, the alpha-1-microglobulin genes or aldolase genes, either in a single copy in the form of several copies thereof and either in an isolated form or in combination with other liver-specific expression elements such as cytomegalovirus, alpha-1-antitrypsin or albumin promoters.

Other examples of promoters which are tissue-specific include the promoter of the albumin gene (Miyatake et al., 1997, J. Virol, 71:5124-32), the core promoter of hepatitis virus (Sandig et al, 1996, Gene Ther., 3:1002-9); the promoter of the alpha-phetoprotein gene (Arbuthnot et al., 1996, Hum.GeneTher., 7:1503-14), and the promoter of the globulin-binding protein which binds to thyroxine (Wang, L., et al., 1997, Proc. Natl. Acad. Sci. USA 94:11563-11566).

The polynucleotides of the invention or the gene constructs forming them can form part of a vector. Thus, in another aspect, the invention relates to a vector comprising a polynucleotide or a gene construct of the invention. A person skilled in the art will understand that there is no limitation as regards the type of vector which can be used because said vector can be a cloning vector suitable for propagation and for obtaining the polynucleotides or suitable gene constructs or expression vectors in different heterologous organisms suitable for purifying the conjugates. Thus, suitable vectors according to the present invention include expression vectors in prokaryotes such as pUC18, pUC19, Bluescript and their derivatives, mp18, mp19, pBR322, pMB9, CoIE1, pCR1, RP4, phages and shuttle vectors such as pSA3 and pAT28, expression vectors in yeasts such as vectors of the type of 2 micron plasmids, integration plasmids, YEP vectors, centromeric plasmids and the like, expression vectors in insect cells such as the pAC series and pVL series vectors, expression vectors in plants such as vectors of expression in plants such as pIBI, pEarleyGate, pAVA, pCAMBIA, pGSA, pGWB, pMDC, pMY, pORE series vectors and the like and expression vectors in superior eukaryotic cells based on viral vectors (adenoviruses, viruses associated to adenoviruses as well as retroviruses and lentiviruses) as well as non-viral vectors such as pSilencer 4.1-CMV (Ambion), pcDNA3, pcDNA3.1/hyg pHCMV/Zeo, pCR3.1, pEF1/His, pIND/GS, pRc/HCMV2, pSV40/Zeo2, pTRACER-HCMV, pUB6/V5-His, pVAX1, pZeoSV2, pCI, pSVL and pKSV-10, pBPV-1, pML2d and pTDT1.

The vector of the invention can be used to transform, transfect or infect cells which can be transformed, transfected or infected by said vector. Said cells can be prokaryotic or eukaryotic. By way of example, the vector wherein said DNA sequence is introduced can be a plasmid or a vector which, when it is introduced in a host cell, is integrated in the genome of said cell and replicates together with the chromosome (or chromosomes) in which it has been integrated. Said vector can be obtained by conventional methods known by the persons skilled in the art (Sambrook et al., 2001, supra.).

Therefore, in another aspect, the invention relates to a cell comprising a polynucleotide, a gene construct or a vector of the invention, for which said cell has been able to be transformed, transfected or infected with a construct or vector provided by this invention. The transformed, transfected or infected cells can be obtained by conventional methods known by persons skilled in the art (Sambrook et al., 2001, supra.). In a particular embodiment, said host cell is an animal cell transfected or infected with a suitable vector.

Host cells suitable for the expression of the conjugates of the invention include, without being limited to, mammal, plant, insect, fungal and bacterial cells. Bacterial cells include, without being limited to, Gram-positive bacterial cells such as species of the Bacillus, Streptomyces and Staphylococcus genus and Gram-negative bacterial cells such as cells of the Escherichia and Pseudomonas genus. Fungal cells preferably include cells of yeasts such as Saccharomyces, Pichia pastoris and Hansenula polymorphs. Insect cells include, without being limited to, Drosophila cells and Sf9 cells. Plant cells include, among others, cells of crop plants such as cereals, medicinal, ornamental or bulbous plants.

In a preferred embodiment, the cell comprising the polypeptide of the invention, the nucleic acid of the invention or the vector of the invention is a human cell. Suitable human cells in the present invention include epithelial cell lines, osteosarcoma cell lines, neuroblastoma cell lines (human, etc.), epithelial carcinomas (human, etc.), glial cells (murine, etc.), hepatic cell lines (from monkey, etc.), COS cells, BHK cells, HeLa cells, 911, AT1080, A549, 293 or PER.C6, NTERA-2 human ECC cells, D3 cells of the mESC line, human stem cells such as HS293 and BGV01, SHEF1, SHEF2 and HS181, NIH3T3 cells, 293T, REH and MCF-7 and hMSC cells.

The adapter polypeptide of the invention may be obtained by recombinant expression in a suitable host. For this purpose, a polynucleotide of the invention is introduced in a vector suitable for its expression in a heterologous organism together with transcription and, optionally, translation control elements. The transcription and, optionally, translation control elements present in the expression cassette of the invention include promoters, which direct the transcription of the nucleotide sequence to which they are operatively linked and other sequences which are necessary or suitable for the transcription and its suitable regulation in time and place, for example, initiation and termination signals, cleavage sites, polyadenylation signal, replication origin, transcriptional enhancers, transcriptional silencers, etc. Said elements, as well as the vectors used for constructing the expression cassettes and the recombinant vectors according to the invention are generally chosen according to the host cells to be used.

Thus, in another aspect, the invention relates to a method of making an adapter protein according to the invention comprising a domain of CAR capable of binding to an adenoviral fiber protein or a functional variant thereof, a trimerization motif, and a human CD40 ligand, the method comprising:
(a) culturing a host cell as defined above under conditions that allow production of the polypeptide; and
(b) isolating the polypeptide.

In a preferred embodiment, the host cell wherein expression is carried out is a human cell. Suitable human cells for producing the polypeptide of the invention include, without limitation, any of the cell lines defined above in connection with the cells of the invention.

Compositions and Complexes of the Invention

The authors of the present invention have shown that DCs can be contacted with an adenoviral particle in the presence of the adapter molecule of the invention in order to promote maturation of the DCs as well as to promote the in vitro and in vivo stimulatory capacity. Thus, compositions comprising an adenoviral particle and an adapter polypeptide of the invention are particularly suited for producing DCs that can be used as DC vaccination. Thus, in another aspect, the invention relates to a composition or complex comprising:
(a) an adapter polypeptide of the invention and
(b) an adenovirus encoding an antigen.

The term “composition”, as used herein, relates to any composition of matter comprising the components of the invention, i.e., the adapter polypeptide of the invention and the adenovirus encoding an antigen. It will be understood that the composition may be formulated as a single component or, alternatively, it can be provided as separated formulations which may then combined for their joint administration. The compositions of the invention may also be provided as a kit-of-parts wherein each of the components is separately formulated but packaged in a single container. The molar ratio of the components forming the compositions of the invention may vary but preferably includes ratios of the two components being between 50:1 and 1:50, more in particular between 20:1 and 1:20, between 1:10 and 10:1, o between 5:1 and 1:5.

The term “complex”, as used herein, relates to compositions of matter wherein one or more adenoviral particles encoding an antigen are bound by one or more molecules of the adapter molecules of the invention via the specific interaction between the CAR domain in the adapter molecule and the adenoviral fiber protein. It will be understood that the stoichiometry of the complex will depend on the number of fiber proteins available on the adenoviral capsid which may bind simultaneously the trimeric adapter protein. The adenoviral capsid is an assembly of seven polypeptides, organized into an icosahedral shell of approximately 900 Å-diameter.

Twelve trimers of hexon, the major capsid component, are arranged onto each of 20 interlocking triangular facets, with penton capsomeres and their protruding fibers occupying each of the 12 vertex positions. Thus, since the adenovirus comprises 12 fibers, the complex of the invention may comprise at the most 12 adapter molecules bound simultaneously to each adenoviral particle. Thus, preferably, the stoichiometry of the complex of the invention is 12 adapter molecules per adenoviral particles, although stoichiometris of 11:1, 10:1, 9:1, 8:1, 7:1, 6:1, 5:1, 4:1, 3:1, 2:1 and 1:1 are also possible and contemplated by the present invention.

The first component of the composition or complex of the invention has been described in detail in the context of the polypeptide of the invention.

The second component of the composition or complex of the invention is an adenovirus encoding an antigen. As used herein, the term “adenovirus” or “adenoviral particle” is used to include any and all viruses that can be categorized as an adenovirus, including any adenovirus that infects a human or an animal, including all groups, subgroups, and serotypes. There are at least 51 serotypes of Adenovirus that classified into several subgroups. For example, subgroup A includes adenovirus serotypes 12, 18, and 31. Subgroup C includes adenovirus serotypes 1, 2, 5, and 6. Subgroup D includes adenovirus serotype 8, 9, 10, 13, 15, 17, 19, 20, 22-30, 32, 33, 36-39, and 42-49. Subgroup E includes adenovirus serotype 4. Subgroup F includes adenovirus serotypes 40 and 41. These latter two serotypes have a long and a short fiber protein. Thus, as used herein an adenovirus or adenovirus particle is a packaged vector or genome. Moreover, the term “adenovirus” and “adenovirus particle” also refer to derivatives thereof containing one or more modifications with respect to the wild-type. Such modifications include, but are not limited to, modifications to the adenovirus genome that is packaged in the particle in order to make an infectious virus. Exemplary modifications include deletions known in the art, such as deletions in one or more of the E1a, E1b, E2a, E2b, E3, or E4 coding regions. Other exemplary modifications include deletions of all of the coding regions of the adenoviral genome. Such adenoviruses are known as “gutless” adenoviruses. The terms also include replication-conditional adenoviruses, which are viruses that preferentially replicate in certain types of cells or tissues but to a lesser degree or not at all in other types. For example, among the adenoviral particles provided herein, are adenoviral particles that replicate in abnormally proliferating tissue, such as solid tumors and other neoplasms. These include the viruses disclosed in U.S. Pat. No. 5,998,205 and U.S. Pat. No. 5,801,029. Such viruses are sometimes referred to as “cytolytic” or “cytopathic” viruses (or vectors), and, if they have such an effect on neoplastic cells, are referred to as “oncolytic” viruses (or vectors).

The adenoviruses forming part of the compositions or complex of the invention comprise a polynucleotide sequence encoding an antigen.

Suitable polynucleotides to be incorporated in the adenoviruses which form the composition and complex of the invention include but are not limited to those encoding all or part of a viral antigen, a bacterial antigen, a fungal antigen, a differentiation antigen, a tumor antigen, an embryonic antigen, an antigen of oncogenes and mutated tumor-suppressor genes, a unique tumor antigen resulting from chromosomal translocations and/or derivatives thereof.

Viral antigens which are capable of eliciting an immune response against the virus include HIV-1 antigens, (such as tat, nef, gp120 or gp160, gp40, p24, gag, env, vif, vpr, vpu, rev), human herpes viruses, (such as gH, gL gM gB gC gK gE or gD or derivatives thereof or Immediate Early protein such as ICP27 , ICP47, ICP4, ICP36 from HSV1 or HSV2, cytomegalovirus, especially Human, (such as gB or derivatives thereof), Epstein Barr virus (such as gp350 or derivatives thereof), Varicella Zoster Virus (such as gp1, II, I11 and IE63), or from a hepatitis virus such as hepatitis B virus (for example Hepatitis B Surface antigen or Hepatitis core antigen), hepatitis C virus (for example core, E1, NS3 or NS5 antigens), from paramyxoviruses such as Respiratory Syncytial virus (such as F and G proteins or derivatives thereof), from parainfluenza virus, from rubella virus (such as proteins E1 and E2), measles virus, mumps virus, human papilloma viruses (for example HPV6, 11, 16, 18, e.g., L1, L2, E1, E2, E3, E4, E5, E6, E7), flaviviruses (e.g. Yellow Fever Virus, Dengue Virus, Tick-borne encephalitis virus, Japanese Encephalitis Virus) or Influenza virus cells, such as HA, NP, NA, or M proteins, or combinations thereof), rotavirus antigens (such as VP7sc and other rotaviral components), and the like (see Fundamental Virology, Second Edition, eds. Fields, B. N. and Knipe, D. M. (Raven Press, New York, 1991) for additional examples of viral antigens)

Bacterial antigens include such as antigens from Neisseria spp, including N. gonorrhea and N. meningitidis (transferrin-binding proteins, lactoferrin binding proteins, PiIC and adhesins); antigens from S. pyogenes (such as M proteins or fragments thereof and C5A protease); antigens from S. agalactiae, S. mutans; H. ducreyi; Moraxella spp, including M catarrhalis, also known as Branhamella catarrhalis (such as high and low molecular weight adhesins and invasins); antigens from Bordetella spp, including B. pertussis (for example parapertussis and B. bronchiseptica (such as pertactin, pertussis toxin or derivatives thereof, filamenteous hemagglutinin, adenylate cyclase, fimbriae); antigens from Mycobacterium spp., including M. tuberculosis, M. bovis, M. leprae, M. avium, M. paratuberculosis, M. smegmatis; Legionella spp, including L. pneumophila; (for example ESAT6, Antigen 85A, -B or -C, MPT 44, MPT59, MPT45, HSPIO,HSP65, HSP70, HSP 75, HSP90, PPD 19 kDa [Rv3763], PPD 38 kDa [Rv0934]) ; antigens from Escherichia spp, including enterotoxic E. coli (for example colonization factors, heat-labile toxin or derivatives thereof, heat-stable toxin or derivatives thereof), antigens from enterohemorragic E. coli and enteropathogenic E. coli (for example shiga toxin-like toxin or derivatives thereof); antigens from Vibrio spp, including V. cholera (for example cholera toxin or derivatives thereof); antigens from Shigella spp, including S. sonnei, S. dysenteriae, S. flexnerii; Yersinia spp, including Y. enterocolitica (for example a Yop protein); antigens from Y. pestis, Y. pseudotuberculosis; Campylobacter spp, including C. jejuni (for example toxins, adhesins and invasins); antigens from Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); antigens from Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof); antigens from C. botulinum (for example botulinum toxin and derivative thereof), antigens from C. difficile (for example clostridium toxins A or B and derivatives thereof); antigens from Bacillus spp., including B. anthracis (for example anthrax toxin and derivatives thereof); Corynebacterium spp., including C. diphtheriae (for example diphtheria toxin and derivatives thereof); antigens from Borrelia spp., including B. burgdorferi (for example OspA, OspC, DbpA, DbpB); antigens from B. garinii (for example OspA, OspC, DbpA, DbpB), B. afzelii (for example OspA, OspC, DbpA, DbpB), antigens from B. andersonfi (for example OspA, OspC, DbpA, DbpB), antigens from B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins); antigens from Chlamydia pneumoniae (for example MOMP, heparin-binding proteins), antigens from C. psittaci; Leptospira spp., including L. interrogans; Treponema spp., including T. pallidum (for example the rare outer membrane proteins), antigens from T. denticola, T. hyodysenteriae; antigens from Plasmodium spp., including P. falciparum; Toxoplasma spp. and T. gondii (for example SAG2, SAGS, Tg34); antigens from Entamoeba spp., including E. histolytica; Babesia spp., including B. microtia; Trypanosoma spp., including T. cruzi; Giardia spp., including G. lamblia; leishmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans; antigens from M. tuberculosis (such as Rv2557, Rv2558, RPFs: Rv0837c, Rv1884c, Rv2389c, Rv2450, Rv1009, aceA (Rv0467), PstS1, (Rv0932), SodA (Rv3846), Rv2031c 16kDal., Tb Ra12, Tb H9, Tb Ra35, Tb38-1, Erd 14, DPV, MTI, MSL, mTTC2 and hTCC1); antigens from Chlamydia (such as the High Molecular Weight Protein (HWMP), ORF3 (EP 366 412), and putative membrane proteins (Pmps); antigens from Streptococcus spp, including S. pneumoniae (PsaA, PspA, streptolysin, choline-binding proteins, the protein antigen Pneumolysin, and mutant detoxified derivatives thereof); antigens derived from Haemophilus spp., including H. influenzae type B (for example PRP and conjugates thereof); antigens from non typeable H. influenzae (such as OMP26, high molecular weight adhesins, P5, P6, protein D and lipoprotein D, and fimbrin and fimbrin derived peptides, or multiple copy variants or fusion proteins thereof); antigens derived from Plasmodium falciparum (such as RTS.S, TRAP, MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.)

Fungal antigens for use in the adenoviruses forming the complexes of the invention include, without limitation, e.g., Candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Protozoal antigens include, but are not limited to, Plasmodium falciparum antigens such as merozoite surface antigens, sporozoite surface antigens, circumsporozoite antigens, gametocyte/gamete surface antigens, blood-stage antigen pf, 55/RESA and other plasmodial antigen components; toxoplasma antigens such as SAG-I, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and Trypanosoma cruzi antigens such as the 75-77 kDa antigen, the 56 kDa antigen and other trypanosomal antigen components.

The antigen can be an allergen or environmental antigen, such as, but not limited to, an antigen derived from naturally occurring allergens such as pollen allergens (tree-, herb, weed, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinoles and platanaceae including La. birch (Betula), alder (Alnus), hazel (Corylus), hornbeam (Carpinus) and olive (Olea), cedar (Cryptomeriaand Juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps and ants (superfamily Formicoidae). Still other allergen antigens that may be used include inhalation allergens from fungi such as from the genera Alternaria and Cladosporium.

The antigen can also be a tumor antigens such as MAGE, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its antigenic epitopes CAP-1 and CAP-2, etv6, am11, Prostate Specific Antigen (PSA) and its antigenic epitopes PSA-1, PSA-2, and PSA-3, prostate-specific membrane antigen (PSMA), T-cell receptor/CD3-ζ chain, MAGE-family of tumor antigens (e.g., MAGE-A1, MAGE-A2, MAGE-A3, MAGEA4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGEC5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, 13-catenin, γ-catenin, p12Octn, gp100^{Pme1117}, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, HA, EBV-encoded nuclear antigen (EBNA)-1, brain glycogen phosphorylase, SSX-1, SSX-2 (HOM-MEL40), SSX-3, SSX-4, SSX-5, SCP-1 and CT-7, and c-erbB-2, acute lymphoblastic leukemia (etv6, amll, cyclophilin b), B cell lymphoma (Ig-idiotype), glioma (E-cadherin, a-catenin,13-catenin, 7-catenin, p12Octn), bladder cancer (p21ras), biliary cancer (p21ras), breast cancer (MUC family, HER2/neu, c-erbB-2), cervical carcinoma (p53, p21ras), colon carcinoma (p21ras, HER2/neu, c-erbB-2, MUC family), colorectal cancer (Colorectal associated antigen (CRC)-0017-1A/GA733, APC), choriocarcinoma (CEA), epithelial cell cancer (cyclophilin b), gastric cancer (HER2/neu, c-erbB-2, ga733 glycoprotein), hepatocellular cancer, Hodgkins lymphoma (lmp-1, EBNA-1), lung cancer (CEA, MAGE-3, NY-ESO-1), lymphoid cell-derived leukemia (cyclophilin b), melanoma (p15 protein, gp75, oncofetal antigen, GM2 and GD2 gangliosides, MelanA/MART-1, cdc27, MAGE-3, p21ras, gp100^{Pme1117}), myeloma (MUC family, p21ras), non-small cell lung carcinoma (HER2/neu, c-erbB-2), nasopharyngeal cancer (lmp-1, EBNA-1), ovarian cancer (MUC family, HER2/neu, c-erbB-2), prostate cancer (Prostate Specific Antigen (PSA) and its antigenic epitopes PSA-1, PSA-2, and PSA-3, PSMA, HER2/neu, c-erbB-2, ga733 glycoprotein), renal cancer (HER2/neu, c-erbB-2), squamous cell cancers of the cervix and esophagus (viral products such as human papilloma virus proteins), testicular cancer (NY-ES0-1), and T cell leukemia (HTLV-1 epitopes).

In a preferred embodiment, the antigenic polypeptide is an HCV antigen. In a still more preferred embodiment, the HCV antigen is the NS3 protein or a fragment thereof.

The HCV NS3 protease corresponds to the HCV polyprotein region spanning amino acids 1027-1657 from any HCV types including, without limitation, HCV genotypes 1, 2, 3, 4, 5, 6, 7, 8, 9, 10, 11 and known subtypes thereof include HCV subtypes 1a, 1b, 1c, 1d, 1e, 1f 1g, 2a, 2b, 2c, 2d, 2e, 2f, 2g, 2h, 2i, 2k, 2l, 3a, 3b, 3c, 3d, 3e, 3f, 3g, 4a, 4b, 4c, 4d, 4e, 4f, 4g, 4h, 4i, 4j, 4k, 4l, 4m, 5a, 6a, 6b, 7a, 7b, 7c, 7d, 8a, 8b, 8c, 8d, 9a, 9b, 9c, 10a and 11 a. It is to be understood that these endpoints are approximations. The mentioned endpoints are not absolute as they may vary, e.g., due to insertions/deletions in an upstream part of the HCV polyprotein or in the HCV NS3 region itself Such insertions/deletions are known to be present as is apparent when HCV polyprotein sequences of different genotypes are compared.

The term “NS3 fragment”, as used herein, relates to a region of HCV NS3 which comprises at least one HCV NS3 epitope (B-cell epitope or T-cell epitope). The term “epitope”, as used herein, means a peptide sequence of at least 3 to 5, preferably about 5 to 10 or 15 and not more than 1,000 amino acids which define a sequence that by itself or as part of a larger sequence, binds to an antibody generated in response to such sequence.

In a specific embodiment, the NS3 antigen of the invention comprises the HCV NS3 peptide spanning amino acids 1188 to 1468 of the HCV polyprotein. Suitable fragments of the NS3 polyprotein which comprise one or more epitopes include, without limitation:
The peptide as defined in NCBI under accession number ACH81020 (SEQ ID NO:12)
The peptide spanning amino acids 1071 to 1084 of the HCV polyprotein region or parts thereof, such as amino acids 1073 to 1081 of the HCV polyprotein region.
The peptide spanning amino acids 1192-1458 comprising the NS3 helicase domain from HVC H,
The peptide spanning amino acids 1406 to 1415 of the HCV polyprotein and variants thereof as described in Table 1 of WO200756760,
The peptide spanning amino acids 1188 to 1468 of the HCV polyprotein,
The c25 NS3 epitope
The epitopes described in U.S. Pat. No. 5,350,671, Chien et al. (Proc. Natl. Acad. Sci. USA, 1992, 89:10011-10015); Chien et al. (J. Gastroent. Hepatol., 1993, 8:S33-39); WO 9300365; WO9401778 and U.S. Pat. No. 6,150,087.
Any CTL and T helper epitopes of HCV NS3 included in the HCV epitope database (http://hcv.lanl.gov/content/immuno/immuno-main.html) (Yusim K, et al., Applied Bioinformatics 2005;4(4).
The peptides identified by Arribillaga et al. (Vaccine, 2002, 21:202-210) having H-2d binding motifs and having the sequences as shown in Table 1.

TABLE 1

| Sequence | Position of first amino acid | SEQ ID NO: |
|---|---|---|
| TGAPVTYSTY | 1048 | 13 |
| VLSTATQSFL | 1062 | 14 |
| KGSSGGPLL | 1164 | 15 |
| LLCPSGHVV | 1170 | 16 |
| FIPVESMETT | 1196 | 17 |
| SSPPAVPQTF | 1215 | 18 |
| AYMSKAHGI | 1267 | 19 |
| RTGVRTITTTG | 1280 | 20 |
| RTITTGGPI | 1284 | 21 |
| TYSTYCKFL | 1293 | 22 |
| TILGIGTVL | 1326 | 23 |
| IGTVLDQAET | 1330 | 24 |
| VALSNTGEI | 1366 | 25 |
| AIPIEAIKGG | 1380 | 26 |
| KCDELAAKLT | 1400 | 27 |
| VVVVATDALM | 1433 | 28 |
| TFTIETTTL | 1460 | 29 |
| VDFSLDPTFT | 1463 | 30 |
| DAVSRAQRRG | 1481 | 31 |
| AYLNTPGLP | 1542 | 32 |
| SVFTGLTHI | 1561 | 33 |
| LTHIDAHFL | 1566 | 34 |
| CLIRLKPTL | 1611 | 35 |
| GPTPLLYRLG | 1621 | 36 |
| LTHPITKYI | 1638 | 37 |

In a preferred embodiment, the NS3 antigen corresponds to the NS3 protein from HCV genotype 1b corresponding to

(SEQ ID NO: 12)
APITAYSQQTRGLLGCIITSLTGRDKNQVDGEVQVLSTATQSFLATCV
NGVCWTVYHGAGSKTLAGPKGPITQMYTNVDQDLVGWPAPPGARSMTP
CTCGSSDLYLVTRHADVVPVRRRGDSRGSLLSPRPISYLKGSSGGPLL
CPSGHVVGIFRAAVCTRGVAKAVDFIPVESMETTMRSPVFTDNSSPPA
VPQTFQVAHLHAPTGSGKSTKVPAAYAAQGYKVLVLNPSVAATLGFGA
YMSKAHGIEPNIRTGVRTITTGGPITYSTYCKFLADGGCSGGAYDIII
CDECHSTDSTTILGIGTVLDQAETAGARLVVLATATPPGSITVPHPNI
EEVALSNTGEIPFYGKAIPTEATKGGRHLIFCHSKKKCDELAAKLTGL
GLNAVAYYRGLDVSVIPTSGDVVVVATDALMTGFTGDFDSVIDCNTCV
TQTVDFSLDPTFTIETTTLPQDAVSPAQRRGRTGRGRSGIERFVTPGE
RPSGMFDSSVLCECYDAGCAWYELTPAETSVRTRAYLNTPGLPVCQDH
LEFWESVFTGLTHIDAHFLSQTKQAGDNLPYLVAYQATVCAPAQAPPP
SWDQMWKCLIRLKPILHGPTPLLYPIGAVQNEVILTHPITKYIMACMS
ADLEVVTSTWV

Pharmaceutical Compositions of the Invention

In another aspect, the invention relates to a pharmaceutical composition comprising a composition or complex of the invention as defined in the previous section and a pharmaceutically acceptable carrier.

As used herein, the term “pharmaceutically acceptable carrier” means a non-toxic, inert solid, semi-solid or liquid filler, diluent, encapsulating material or formulation auxiliary of any type. Remington's Pharmaceutical Sciences. Ed. by Gennaro, Mack Publishing, Easton, Pa., 1995 discloses various carriers used in formulating pharmaceutical compositions and known techniques for the preparation thereof. Some examples of materials which can serve as pharmaceutically acceptable carriers include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN™ 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. If filtration or other terminal sterilization methods are not feasible, the formulations can be manufactured under aseptic conditions.

Under certain conditions, it may be preferable to provide the complex or composition of the invention as a controlled release formulation. The term “controlled release” (and variants of that term) as used herein (e.g., in the context of “controlled-release system”) is generally meant to encompass release of a substance (e.g., a drug or a protein) at a selected site or otherwise controllable in rate, interval, and/or amount. Controlled release encompasses, but is not necessarily limited to, substantially continuous delivery, patterned delivery (e.g., intermittent delivery over a period of time that is interrupted by regular or irregular time intervals), and delivery of a bolus of a selected substance (e.g., as a predetermined, discrete amount if a substance over a relatively short period of time (e.g., a few seconds or minutes).

Therapeutic Methods of the Invention

The authors of the present invention have observed that the administration to a subject of a DC previously contacted with an adenoviral particle encoding an antigen in the presence of an adapter of the invention results in a response against isolated NS3 antigens which is more potent than that observed with DCs contacted with the adenoviral particle in the absence of the adapter molecule. This result allows the use of the complexes of the invention both for direct administration to a subject in need thereof whereby the subject's own DCs will be transduced in vivo by the adenoviral particles.

Thus, in another aspect, the invention relates to a method of eliciting an immune response against an antigen in a subject comprising the steps of administering to the subject a composition or complex comprising:
(a) an adapter polypeptide of the invention and
(b) an adenovirus encoding an antigen.

In another aspect, the invention relates to a composition or complex comprising:
(a) an adapter polypeptide of the invention and
(b) an adenovirus encoding an antigen,
   or a pharmaceutical composition comprising said composition or complex of the invention for use in medicine.

In another aspect, the invention relates to the use of a composition or complex comprising
(a) an adapter polypeptide of the invention and
(b) an adenovirus encoding an antigen,
   or a pharmaceutical composition comprising said composition or complex of the invention for the manufacture of a medicament for inducing an immune response against said antigen.

In another aspect, the invention relates to a composition or complex comprising:
(a) an adapter polypeptide of the invention and
(b) an adenovirus encoding an antigen,
   or a pharmaceutical composition comprising said composition or complex of the invention for inducing an immune response against said antigen.

Components (a) and (b) of the complex used in the method of the invention are essentially those as described in detail in the context of the complexes of the invention and need not be further described. In a preferred embodiment, the adenovirus encodes HCV antigen in which case the method is used for the treatment or prevention of hepatitis C. In a still more preferred embodiment, the HCV antigen is an NS3 protease or an antigenic fragment thereof.

In a preferred embodiment, the complex is formed prior to the administration by contacting the components of the complex under conditions adequate for the formation of said complex. The conditions which are adequate for the formation of the complex can be easily determined by one of ordinary skill in the art using conventional techniques for determination of the association of two components such as non-reducing SDS-PAGE gradient centrifugation, chromatography, bioluminescence resonance energy transfer (BRET), fluorescence resonance energy transfer (FRET) and the like.

The complexes of the invention can be administered to a patient by any means known in the art including oral and parenteral routes. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

The complexes may be administered to a subject in need thereof systemically, e.g., by IV infusion or injection. Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be suppositories which can be prepared by mixing the inventive conjugate with suitable non-irritating excipients or carriers such as cocoa butter, polyethylene glycol, or a suppository wax which are solid at ambient temperature but liquid at body temperature and therefore melt in the rectum or vaginal cavity and release the inventive conjugate.

Dosage forms for topical or transdermal administration of an inventive pharmaceutical composition include ointments, pastes, creams, lotions, gels, powders, solutions, sprays, inhalants, or patches. The inventive conjugate is admixed under sterile conditions with a pharmaceutically acceptable carrier and any needed preservatives or buffers as may be required. Ophthalmic formulations, ear drops, and eye drops are also contemplated as being within the scope of this invention. The ointments, pastes, creams, and gels may contain, in addition to the inventive conjugates of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof. Transdermal patches have the added advantage of providing controlled delivery of a compound to the body. Such dosage forms can be made by dissolving or dispensing the inventive conjugates in a proper medium. Absorption enhancers can also be used to increase the flux of the compound across the skin. The rate can be controlled by either providing a rate controlling membrane or by dispersing the inventive conjugates in a polymer matrix or gel. Powders and sprays can contain, in addition to the inventive conjugates of this invention, excipients such as lactose, talc, silicic acid, aluminum hydroxide, calcium silicates, and polyamide powder, or mixtures thereof. Sprays can additionally contain customary propellants such as chlorofluorohydrocarbons. When administered orally, the inventive complex of the invention can be, but are not necessarily, encapsulated. A variety of suitable encapsulation systems are known in the art (“Microcapsules and Nanoparticles in Medicine and Pharmacy,” Edited by Doubrow, M., CRC Press, Boca Raton, 1992; Mathiowitz and Langer J. Control. Release 5:13, 1987; Mathiowitz et al. Reactive Polymers 6:275, 1987; Mathiowitz et al. J. Appl. Polymer Sci. 35:755, 1988; Langer

Ace. Chem. Res. 33:94,2000; Langer J. Control. Release 62:7,1999; Uhrich et al. Chem. Rev. 99:3181,1999; Zhou et al. J. Control. Release 75:27, 2001; and Hanes et al. Pharm. Biotechnol. 6:389,1995). The inventive conjugates may be encapsulated within biodegradable polymeric microspheres or liposomes. Examples of natural and synthetic polymers useful in the preparation of biodegradable microspheres include carbohydrates such as alginate, cellulose, polyhydroxyalkanoates, polyamides, polyphosphazenes, polypropylfumarates, polyethers, polyacetals, polycyanoacry lates, biodegradable polyurethanes, polycarbonates, polyanhydrides, polyhydroxyacids, poly(ortho esters), and other biodegradable polyesters. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides.

Pharmaceutical compositions for oral administration can be liquid or solid. Liquid dosage forms suitable for oral administration of inventive compositions include pharmaceutically acceptable emulsions, microemulsions, solutions, suspensions, syrups, and elixirs. In addition to an encapsulated or unencapsulated conjugate, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants, wetting agents, emulsifying and suspending agents, sweetening, flavoring, and perfuming agents. As used herein, the term “adjuvant” refers to any compound which is a nonspecific modulator of the immune response. In certain embodiments, the adjuvant stimulates the immune response. Any adjuvant may be used in accordance with the present invention. A large number of adjuvant compounds are known in the art (Allison Dev. Biol. Stand. 92:3-11, 1998; Unkeless et al. Annu Rev. Immunol. 6:251-281,1998; and Phillips et al. Vaccine 10:151-158,1992).

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. It will be appreciated that the exact dosage of the targeted reverse micelle particle is chosen by the individual physician in view of the patient to be treated, in general, dosage and administration are adjusted to provide an effective amount of the targeted particle to the patient being treated. As used herein, the “effective amount” of a targeted particle refers to the amount necessary to elicit the desired biological response. As will be appreciated by those of ordinary skill in this art, the effective amount of targeted particle may vary depending on such factors as the desired biological endpoint, the drug to be delivered, the target tissue, the route of administration, etc. For example, the effective amount of targeted particle containing an anti-cancer drug might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

The complexes of the invention may be formulated in dosage unit form for ease administration and uniformity of dosage. The expression “dosage unit form” as used herein refers to a physically discrete unit of complex appropriate for the patient to be treated. It will be understood, however, that the total daily usage of the compositions of the present invention will be decided by the attending physician within the scope of sound medical judgment. For any complex, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of complex can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices may be useful in some embodiments. The data obtained from cell culture assays and animal studies can be used in formulating a range of dosage for human use.

The complexes are delivered at a dose ranging from approximately 1 complex per kilogram of body weight to approximately 10¹⁴ complexes per kilogram of body weight. Generally, they are delivered at a dose of approximately 10⁶ complexes per kilogram of body weight to approximately 10¹³ complexes per kilogram of body weight, and typically the dose ranges from approximately 10⁸ complexes per kilogram of body weight to approximately 10¹² complexes per kilogram of body weight.

At selected intervals, DCs from the recipient's lymphoid organs may be used to measure expression, for example, by observing expression of marker genes. T cells from lymph nodes and spleens of virus-treated recipients may be measured from the magnitude and durability of response to antigen stimulation. Tissue cells other than DCs, such as epithelial cells and lymphoid cells, may be analyzed for the specificity of in vivo gene delivery.

Method for Obtaining Antigen-Loaded CD40-Positive Cells

The authors of the present invention have observed that the adapter protein of the inventions allows the efficient transduction of CD40 cells by adenoviruses while at the same time promote maturation of the CD40 due to the interaction of the CD40L moiety of the adapter protein with CD40 in the target cells. In fact, the results provided in examples 1, 2, 3, 4, 8, 9, 10, 11, and 12 disclose the response of DCs to an adenovirus in the presence of an adapter molecule of the invention. Thus, in another aspect, the invention relates to a method of obtaining an antigen-loaded CD40-positive antigen-presenting cell, comprising the steps of
(i) contacting a CD40-positive antigen-presenting cell with an adapter protein of the invention and an adenovirus encoding an antigen, wherein said contacting can be carried out by separately adding the polypeptide and the adenovirus or by adding a preformed polypeptide-adenovirus complex,
(ii) maintaining the mixture obtained in step (i) under conditions adequate for the formation of a ternary complex between said polypeptide, said adenovirus and said cell and
(iii) maintaining the cells under conditions adequate for internalization, processing and presentation of one or more peptides derived from the antigen.

The term “CD40-positive antigen-presenting cell”, as used herein, will be understood to include any cell that can present peptides in the context of MHC molecules and which shows expression of CD40. CD40-positive APCs include, but are not limited to, macrophages, B-cells and dendritic cells, such as immature dendritic cells, mature dendritic cells, plasmacytoid dendritic cells, Langerhans cells and artificial antigen presenting cells.

In a preferred embodiment, the “CD40-positive antigen-presenting cell” is a dendritic cell. As used herein, “dendritic cell” refers to any member of a diverse population of morphologically similar cell types found in lymphoid or non-lymphoid tissues. DCs are referred to as “professional” antigen presenting cells, and have a high capacity for sensitizing MHC-restricted T cells. DCs may be recognized by function, by phenotype and/or by gene expression pattern, particularly by cell surface phenotype. These cells are characterized by their distinctive morphology, high levels of surface MHC-class II expression and ability to present antigen to CD4⁺ and/or CD8⁺ T cells, particularly to naive T cells. CD4⁻ cells activated by dendritic cells produce IFN-.gamma. and induce proliferation and antibody production of antigen-specific B lymphocytes. CD8⁺ T activated by dendritic cells kill cells displaying antigen (such as virus-infected cells) by releasing cytotoxic granules into the cell.

Morphologically, dendritic cells are characterized by an unusual surface, with characteristic vein-like projections, and is characterized by expression of the cell surface markers CD11c and MHC class II. Most DCs are negative for markers of other leukocyte lineages, including T cells, B cells, monocytes/macrophages, and granulocytes. Subpopulations of dendritic cells may also express additional markers including 33D1, CCR1, CCR2, CCR4, CCR5, CCR6, CCR7, CD1a-d, CD4, CD5, CD8alpha, CD9, CD11b, CD24, CD40, CD48, CD54, CD58, CD80, CD83, CD86, CD91, CD117, CD123 (IL3R.alpha.), CD134, CD137, CD150, CD153, CD162, CXCR1, CXCR2, CXCR4, DCIR, DC-LAMP, DC-SIGN, DEC205, E-cadherin, Langerin, mannose receptor, MARCO, TLR2, TLR3 TLR4, TLR5, TLR6, TLR9, and several lectins. The patterns of expression of these cell surface markers may vary along with the maturity of the dendritic cells, their tissue of origin, and/or their species of origin. Functionally, DCs may be identified by any convenient assay for determination of antigen presentation. Such assays may include testing the ability to stimulate antigen-primed and/or naive T cells by presentation of a test antigen, followed by determination of T cell proliferation, release of IL-2, and the like.

In a first step of the method for obtaining antigen-loaded CD40-positive antigen-presenting cell, the CD40-positive antigen-presenting cell is contacted with an adapter protein of the invention and an adenovirus encoding an antigen, wherein said contacting can be carried out by separately adding the polypeptide and the adenovirus or by adding a preformed polypeptide-adenovirus complex.

CD40-positive antigen-presenting cell are first obtained using standard methods from suitable sources. Such suitable tissue sources include, e. g., peripheral blood, bone marrow, tumor-infiltrating cells, peritumoral tissues-infiltrating cells, lymph node biopsies, thymus, spleen, skin, umbilical cord blood, monocytes harvested from peripheral blood, CD34 or CD14 positive cells harvested from peripheral blood, blood marrow or any other suitable tissue or fluid.

In the particular case that the APC is a DC, and due to the fact that it has been observed that patients suffering certain diseases have reduced function of dendritic cells (i. e. defective antigen presentation and defective maturation), it is preferred to obtain precursor cells and then allow then to differentiate in vitro to obtain functional dendritic cells.

Thus, it is contemplated in the present invention that stem cell precursor stimulated dendritic cell differentiation is used as a method for ex vivo treatment of hyperproliferative disease. A method of culturing and inducing the differentiation of monocytes into dendritic cells has been described in U.S. Pat. No. 5,849,589. The method involves culturing the precursor cells in a medium containing with GM-CSF, IL-4 and TNFα. An alternate method of isolating dendritic cells has been described in U.S. Pat. No. 5,643,786. This method involves elutriating peripheral blood samples in at least four flow rates from an elutriation rotor. Calcium ionophore is used to stimulate monocytes isolated during the process into dendritic cells and treatment for diseases involving re-introduction of the activated dendritic cells are also disclosed. It is also possible to prepare immortalized precursor cells that is considered useful in the present invention (U.S. Pat. No. 5,830,682 and U.S. Pat. No. 5,811,297). In another example, an immature dendritic cell line derived from p53 growth suppressor gene deficient animals are prepared (U.S. Pat. No. 5,648,219). The immature dendritic cell line may be induced to become an activated, immortalized dendritic cell line that will stimulate T-cell proliferation and is thus contemplated for use in the present invention.

Once the DCs are available, the cells are then contacted either with an adapter polypeptide of the invention and an adenovirus encoding an antigen or with a preformed complex of the adapter polypeptide and the adenovirus. In the case where the three components are contacted, a ternary complex must be formed resulting from the interaction of the fiber protein of the adenovirus with the CAR domain of the adapter protein and the interaction between the human CD40L in the adapter and the CD40-positive cell. If the complex between the adenovirus and the adapter polypeptide is already preformed, the contacting step requires the formation of a secondary complex between the adenovirus-adapter complex and the CD40-positive cell mediated by the interaction between the human CD40L present in the adenovirus-adapter complex and CD40 present in the surface of the CD40 cell.

The antigen encoded by the adenovirus used in the first step can be any suitable antigen as defined above. In a preferred embodiment, the antigen is an HCV antigen. In a still more preferred embodiment, the HCV antigen is an NS3 protease or an antigenic fragment thereof. Any NS3 variant or antigenic fragment thereof as defined above is suitable for use in the method of the present invention.

In another preferred embodiment, the dendritic cells used in the first step are obtained from an individual suffering from hepatitis C. Contrary to the evidences in the prior art that DCs from patients infected by HCV mature poorly in response to conventional stimuli, the authors of the present invention have made the surprising observation that DCs from HCV-infected patients mature in response to the adapter of the invention similarly as cells from control subjects, as determined by IL-12 expression and their capacity to stimulate allogeneic T lymphocytes (see example 12).

These cells are transduced in vivo with recombinant adenoviral vectors (rAds) expressing a HCV antigen in the presence of an adapter molecule according to the invention.

In a further step, the cells are maintained under conditions adequate for internalization, processing and presentation of one or more peptides derived from the antigen. The conditions suitable for internalization, processing and presentation of at least one antigenic peptide derived from the antigen encoded by the adenovirus can be determined by using standard assays for determining DC activation.

The maturation of DCs can be followed using a number of molecular markers and of cell surface phenotypic alterations. These changes can be analyzed, for example, using flow cytometry techniques. Typically, the maturation markers are labeled using specific antibodies and DCs expressing a marker or a set of markers of interest can be separated from the total DC population using, for example, cell sorting FACS analysis. Markers of DC maturation include genes that are expressed at higher levels in mature DCs compared to immature DCs. Such markers include, but are not limited to, cell surface MHC Class II antigens (in particular HLA-DR), costimulating molecules such as CD40, CD80, CD86, CD83, cell trafficking molecules such as CD54, CD11c and CD18, etc. Moreover, maturation of DCs can be carried out by determining the expression of certain Notch ligands such as Delta-like ligand 4 (DLL4), Jagged 1 and Jagged 2 which are associated with the induction of Th1 responses. Furthermore, mature dendritic cell can be identified based on their ability to stimulate the proliferation of naive allogeneic T cells in a mixed leukocyte reaction (MLR). In addition, it has been shown that, in general, while immature dendritic cells are very efficient at antigen uptake but are poor antigen presenting cells, mature dendritic cells are poor at antigen uptake but are very efficient antigen presenting cells. The antigen presenting function of a dendritic cell can be measured using antigen-dependent, MHC-restricted T cell activation assays as described herein, as well as other standard assays well known to those of skill in the art such as the in vitro stimulatory capacity on peripheral blood lymphocytes, for instance, by the determination of the amount of IFN-γ produced by CD8+lymphocytes in the presence of the DCs. This determination can be carried out using a technique known as ELISPOT. T cell activation can further be determined, e.g., by measuring the induction of cytokine production by the stimulated dendritic cells. The stimulation of cytokine production can be quantifiedquantified using a variety of standard techniques, such as ELISA, well known to those of skill in the art.

Other cytotoxicity assays such as the labeling of target cells with tritiated thymidine (3H-TdR) may also be used. 3H-TdR is taken up by target cells into the nucleus of the cell. Release of 3H-TdR is a measure of cell death by DNA fragmentation. The assay is conducted as above except the incubation period is at least about 48 hours and 50 p, 1 to about 100 ml of the supernatant is measured by a beta-counter in the presence of at least about 1 ml of scintillation fluid. Calculation of percent specific lysis is performed using the above formula.

Any of the dendritic cell preparations of this invention (precursors or mature, immunogenic or toleragenic, and if immunogenic, before or after loading with antigen) can be stored after preparation to be used later for therapeutic administration or further processing. Methods of cryopreserving dendritic cells both before and after loading are described in PCT publication WO0216560.

Dendritic Cell Vaccination

The invention provides a method for obtaining mature and antigen loaded APCs. Thus, in another aspect, the invention relates to an antigen-loaded CD40-positive antigen-presenting cell obtained by the method as defined in the previous section.

The cells may be used for eliciting an immune response in a patient by using them as a DC vaccination, i.e. by the administration to said patient of the cells. Thus, in another aspect, the invention relates to an antigen-loaded CD40-positive antigen-presenting cell as defined in the invention, for eliciting an immune response in a subject. In other words, the invention also relates to a method for eliciting an immune response in a subject comprising administration to a subject of the antigen presenting cell.

The DC vaccination is carried out by the administration of the antigen-loaded DCs into a subject (e.g., human patient) where they induce an immune response. Typically the immune response includes a CTL response against target cells bearing target antigenic peptides (e.g., in a MHC class I/peptide complex). These target cells are typically cancer cells. When the modified DCs are to be administered to a patient, they are preferably isolated or derived from precursor cells from that patient (i.e., the DCs are administered to an autologous patient). However, the cells may be infused into HLA-matched allogeneic, or HLA-mismatched allogenic patients. In the latter case, immunosuppressive drugs may be administered to the recipient.

The cells may be administered in any suitable manner, preferably with a pharmaceutically acceptable carrier (e.g., saline). Usually administration will be intravenous, but intra-articular, intramuscular, intradermal, intraperitoneal, and subcutaneous routes are also acceptable. Administration (i.e., immunization) may be repeated at time intervals. Infusions of DC may be combined with administration of cytokines that act to maintain DC number and activity (e.g., GM-CSF, IL-12).

The dose administered to a patient should be sufficient to induce an immune response as detected by assays which measure T cell proliferation, T lymphocyte cytotoxicity, and/or effect a beneficial therapeutic response in the patient over time. Typically, 10⁶ to 10⁹ or more DCs are infused, if available. The vaccines can be administered one or more times to a patient to impart beneficial results. One skilled in the art will be able to determine the appropriate timing for administering the vaccine. The timing of the first and/or subsequent dose(s) of the vaccine can depend on a variety of factors, including, but not limited to a patient's health, stability, age, and weight. The vaccine can be administered at any appropriate time interval; for example, including but not limited to, once per week, once every two weeks, once every three weeks, once per month. In one embodiment, the vaccine can be administered indefinitely. In one embodiment, the vaccine can be administered three times in two week intervals. Appropriate dosages of the vaccines also depend on a variety of factors, including, but not limited to, a patient's health, stability, age, and weight. Once a sufficient level of immunity has been achieved to achieve clinical benefit, maintenance boosters may be required, but can generally be given on a less frequent basis (e.g., monthly or semi-annually).

The DCs used in the method for eliciting an immune response are preferably formulated so that they can be used as an off-the-shelf pharmaceutical. In this case, there may be a histocompatibility mismatch between the cells in the preparation and the patient being treated. In some instances, mismatch at the Class II loci may enhance the effect of the vaccine. Allogeneic cells can cross-feed host antigen presenting cells by way transferring packaged tumor antigen to them in the form of exosomes (S. L. Alfieri et al., J. Immunother. 27:282, 2004; F. Andre et al., J. Immunol. 172:2126, 2004; N. Chaput et al., Cancer Immunol. Immunother. 53:234, 2004). If the administered cells are taken up instead by phagocytic cells in the host, their tumor antigen payload will be presented by the host cells as a matter of course. In other instances, HLA mismatch may dampen the effect of the vaccine—either by promoting premature elimination of the cells (especially after multiple administration), or by generating a strong anti-allotype response that distracts the immune system from the intended target. In this context, it may be advantageous to use a vaccine preparation in which at least some of the HLA Class I alleles on the dendritic cells (especially at the A locus and, more in particular, the A2 allele) are shared with the patient. In this way, at least some of the tumor target antigen will be presented in autologous Class I molecules, enhancing the anti-tumor response and diminishing the allo response.

Partial match can be achieved simply by providing a dendritic cell vaccine made of a mixture of cells bearing two or more of the common HLA-A allotypes (HLA-A2, A1, A19, A3, A9, and A24). Complete match for most patients can be achieved by providing the clinician with a battery of different dendritic cells from which to select, each possibly bearing only a single allotype at the HLA-A locus. Treatment would involve identifying one or more HLA allotype(s) in the patient by standard tissue typing, and then treating the patient with dendritic cells having HLA allotype(s) that match those of the patient. For example, a patient that was HLA-A2 and A19 could be treated with either HLA-A2 or HLA-A19 homozygous cells, or with a mixture of both.

Potential negative effects of HLA mismatch can also be dealt with by generating immune tolerance against the foreign allotypes. During preparation of the vaccine, the DCs are divided into two populations: one for generating immature toleragenic dendritic cells, and the other for generating mature dendritic cells for antigen presentation. Because they are derived from the same line, the toleragenic cells are designed to induce HLA-specific tolerance that will enhance graft acceptance of the mature cells. The subject first receives one or more administrations of the toleragenic cells to generate a sufficient degree of immune unresponsiveness (measurable, for example, in a mixed lymphocyte reaction). Once tolerance is in place (a week to a month later), the subject is then administered with the antigen-loaded dendritic cells as often as needed to elicit the immune response against the target tumor antigen.

In a preferred embodiment, the antigen presenting cell is a dendritic cell autologous to the subject to be treated.

The DC vaccine compositions may comprise, in addition to the antigen-loaded APCs, an immunstimulatory compound such as a toll-like receptor (TLR) agonist and/or one or more immunostimulatory cytokine. Suitable TLR agonists include, without limitation, agonists acting trough TLR1, TLR2 agonists such as phenol-soluble modulins, TLR3 agonists such as polyinosinic-polycytidylic acid (Poly IC), TLR4 agonists such as one or more of the EDA domain of fibronectin, or a bacterial lipopolysaccharide, a TLR-5 agonist such as bacterial flagellin, a TLR-6 agonist such as mycobacterial lipoprotein, di-acylated LP, and phenol-soluble modulin, a TLR7 agonist such as loxoribine or an imidazoquinoline compound, a TLR-8 agonist such as resiquimod, a TLR-9 agonist such as a polynucleotide containing unmethylated CpG nucleotides.

The term “immunostimulatory cytokine”, as used herein, is understood as any compound which promotes an increase in the activity of any component of the immune system including those components forming part or being involved in cell-mediated immune response, humoral-mediated immune response and the complement system. Preferably, the immunostimulatory cytokine is selected from the group of IL-12, IL-2, IL-15, IL-18, IL-24, GM-CSF, TNFa, CD40 ligand, IFNα, IFNβ, IFNγ and functionally equivalent variants thereof.

The vaccine compositions optionally include an adjuvant. The adjuvant component can be any suitable adjuvant or combination of adjuvants. For example, suitable adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvants (CFA) and Incomplete Freunds Adjuvant (IFA); mineral gels; block copolymers; Avridine™ lipid-amine; SEAM62; adjuvants formed from bacterial cell wall components such as adjuvants including lipopolysaccharides (e.g., lipid A or monophosphoryl lipid A (MPL), trehalose dimycolate (TDM), and cell wall skeleton (CWS); heat shock protein or derivatives thereof; adjuvants derived from ADP-ribosylating bacterial toxins, including diphtheria toxin (DT), pertussis toxin (PT), cholera toxin (CT), the E. coli heat-labile toxins (LT1 and LT2), Pseudomonas endotoxin A, Pseudomonas exotoxin S, B.cereus exoenzyme, B. sphaericus toxin, C. botulinum C2 and C3 toxins, C. limosum exoenzyme, as well as toxins from C. perfringens, C. spiriforma and C. difficile, S. aureus EDIN, and ADP-ribosylating bacterial toxin mutants such as CRM 197, a non-toxic diphtheria toxin mutant; saponin adjuvants such as Quil A (U.S. Pat. No. 5,057,540), or particles generated from saponins such as ISCOMs (immunostimulating complexes); chemokines and cytokines, such as interleukins (e.g., IL-I IL-2, IL-4, IL-5, IL-6, IL-7, IL-8, IL-12, etc.), interferons (e.g., gama interferon), macrophage colony stimulating factor (M-CSF), tumor necrosis factor (TNF), defensins 1 or 2, RANTES, MIP1 -.alpha, and MEP-2, etc; muramyl peptides such as N-acetyl-murarnyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1 ‘-2’-dipalmitoyl-s-n-glycero-3 huydroxyphosphoryloxy)-ethylamine (MTP-PE) etc; adjuvants derived from the CpG family of molecules, CpG dinucleotides and synthetic oligonucleotides which comprise CpG motifs, limosum exoenzyme and synthetic adjuvants such as PCPP.

In the particular case of DC-based vaccines wherein the DCs comprise one or more antigenic peptides of HCV and, more in particular, of the HCV NS3, the vaccines are used for the treatment or prevention of HCV infection as well as for the treatment of other conditions resulting from HCV infection such as asymptomatic chronic carriage, acute hepatitis, chronic hepatitis, cirrhosis, hepatocellular carcinoma and the like.

The invention is described hereinafter by the following examples which are to be construed as merely illustrative and in no case as limitative of the scope of the invention.

### EXAMPLES

### Example 1

The use of CFm40L increases the efficacy of transduction of DCs with adenovirus. Dendritic cells (DCs) of C57BL6 mice were generated as described in Zabaleta et al. (Mol.Ther., 2008, 16:210-217) from bone marrow precursors. To that end, femur and tibia marrow was extracted and the red blood cells were lysed using a lysis buffer solution (0.15 M NH₄Cl, 10 mM KHCO₃, 0.1 mM Na₂EDTA). Washing with RPMI 1640 was then carried out and the lymphocytes and granulocytes were removed by means of incubating with a mixture of antibodies against the different cell populations together with rabbit supplement:
Anti-CD4 antibody (100 μg/ml): Obtained from hybridoma GK1-5 (Dialynas et al., 1983, J Immunol. 1983 November; 131:2445-51).
Anti-CD8 antibody (100 μg/ml): Obtained from rat hybridoma H35.17.2 (Pierres et al., 1982, Eur. J. Immunol. 12 (1982), 60-69.).
Ly-6G/Gr1 (BD Pharmingen; San Diego, Calif.) at 10 μl/ml.
CD45R/B220 (BD Pharmingen) at 15 μl/ml.
Rabbit supplement (SIGMA) at 50 μg/ml.

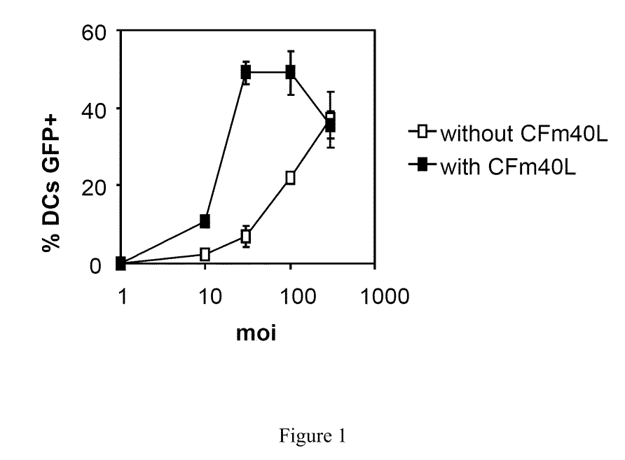

This mixture was incubated at 37° C. for 50 minutes, stirring every 20 minutes. After the incubation, washing was carried out and the resulting cells were cultured at a concentration of 10⁶ cells/ml in 12-well plates (Iwaki, Japan) in complete medium (CM; RPMI 1640 with 10% fetal bovine serum, penicillin (50 U/mL), streptomycin (50 μg/mL), HEPES (5 mM) and glutamine (2 mM)) supplemented with 20 ng/ml of mouse GM-CSF and 20 ng/ml of interleukin-4 (IL-4) (both from Peprotech, London, United Kingdom). Every two days, two thirds of the medium were replaced by fresh medium supplemented with cytokines. On day seven, the non-adherent cells were collected and resuspended in RPMI 1640 at a concentration of 10⁷ cells/ml. Solutions with different amounts of AdGFP adenovirus (encoding the green fluorescent protein) were incubated in the presence or absence of 6 μg of adapter CFm40L in 50 μl of PBS for 30 minutes at 37° C. and then they were added to the DCs (10⁶). The adapter CFm40L was obtained by means of purification from the culture supernatant of 293 cells stably transfected with a plasmid expressing said molecule, as described in Pereboev et al. (Mol. Ther. 2004; 9:712-720) After one hour of incubation, CM supplemented with cytokines was added until diluting the cells to a final concentration of 10⁶ cells/ml. The cells were collected 24 hours later and thoroughly washed in order to completely remove adenoviral particles. After the washing, the cells obtained were used for their analysis by flow cytometry. The results are represented in FIG. 1 as the percentage of transduced cells (GFP⁺) for each of the amounts of virus used.

### Example 2

The transduction of DCs with AdNS3 in the presence of CFm40L induces their in vitro maturation: expression of surface markers. DCs from C57BL6 mice were prepared as described in Example 1 and transduced with

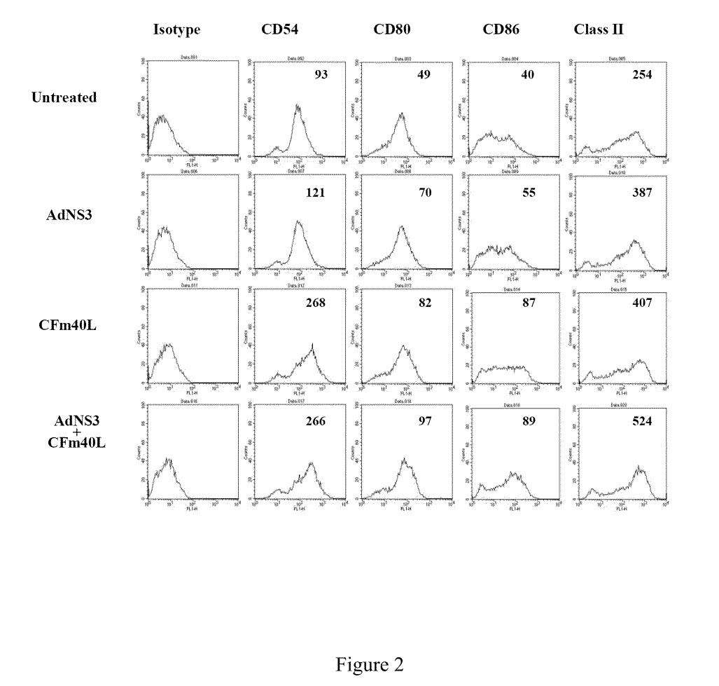

AdNS3 (moi 30) in the presence or absence of CFm40L. Untreated DCs or DCs treated with CFm40L alone were used as control groups. The DCs were collected one day after and their degree of maturation was studied by means of surface marker analysis by flow cytometry. Antibodies against the markers CD54, CD80, CD86, I-Ab (MHC class II), as well as a control isotype (all of them from BD Pharmingen) were used. The labeling was performed at 4° C. in PBS with 2% FBS. After 30 minutes, the cells were washed and the expression of the different surface markers was analyzed. The results are shown in FIG. 2.

### Example 3

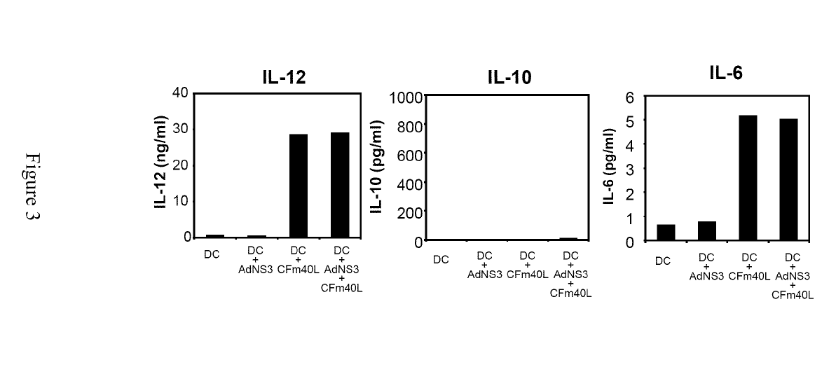

The transduction of DCs with AdNS3 in the presence of CFm40L induces their in vitro maturation: production of cytokines DCs prepared as in example 2 and divided into the same groups (untreated, AdNS3, CFm40L and CFm40L+AdNS3) were cultured for 24 hours and then the culture supernatants were collected. The amount of IL-12, IL-10 and IL-6 produced was determined in these culture supernatants by means of ELISA (BD-Pharmingen, Franklin Lakes, N.J., USA, according to the manufacturer's instructions. The results are shown in FIG. 3.

### Example 4

The maturation of DCs induced by CFm40L is accompanied by the expression of Notch ligands associated to the induction of Th1 responses. DCs were prepared as in Example 2 and transduced with AdNS3 (moi 30) in the presence or absence of CFm40L. The cells were collected one day later and the expression of mRNA of the genes of the Notch ligands Delta-like ligand 4 (DLL4), Jagged 1 and Jagged 2 was analyzed by means of real-time PCR, as described in Zabaleta A et al (Mol Ther. 2008 16:210-7). The primers used for the amplification are shown in the Table:

| Primer | Sequence | SEQ ID NO: |
|---|---|---|
| DLL4 sense | GTGGGTAAGATTTGGCGAAC | 38 |
| DLL4 antisense | GTGGGGGATACATTCATTGC | 39 |
| Jagged 1 sense | TATCTGTCCACCTGGCTATG | 40 |
| Jagged 1 antisense | AGTCACTGGCACGATTGTAG | 41 |
| Jagged 2 sense | TCGTCGTCATTCCCTTTCAG | 42 |
| Jagged 2 antisense | GTGGCACTGTAGTAGTTCTC | 43 |

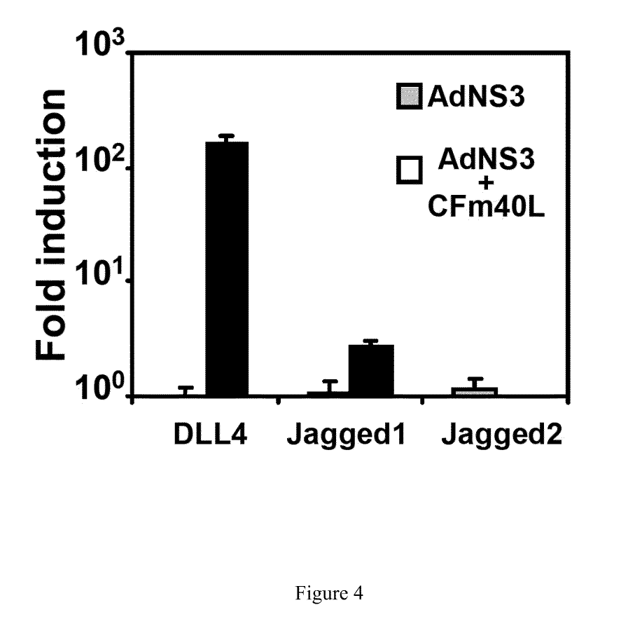

The results were standardized with actin and represented in FIG. 4 as the degree of induction relative to untreated DCs.

### Example 5

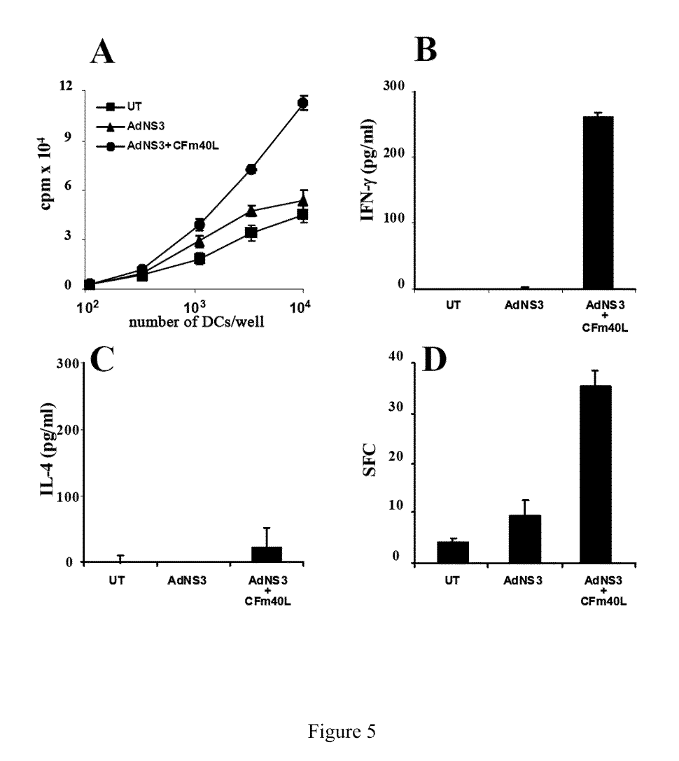

The transduction of DCs with AdNS3 in the presence of CFm40L increases their in vitro stimulatory capacity. Different numbers of DCs from C57BL6 mice subjected to treatments mentioned in examples 2 and 3 (untreated, AdNS3 and CFm40L+AdNS3) were cultured together with allogeneic lymphocytes (10⁵ non-adherent cells obtained from the spleen of BALB/c mice). The assays were performed in a U-bottom 96-well plate and the culture supernatants were collected two days later and 0.5 μCi of [³H] thymidine were added per well, which was left for another 18 hours. (A) After that time, the samples were collected in Unifilter plates (PerkinElmer, Belgium). Once dried, scintillation fluid was added to the plate and the incorporated thymidine was measured in a scintillation counter (Topcount; Packard, Meriden, Conn.). The amount of IFN-gamma (B) and IL-4 (C) present in the supernatants was also determined by means of ELISA (BD Pharmingen). (D) DCs from HHD mice [15] transgenic for the human molecules HLA-A2 and a2-microglobulin were prepared. They were transduced with AdNS3 at a moi of 30 in the presence or absence of CFm40L or they were left untreated and were collected 24 hours later. These DCs (5×10³)/well were used to stimulate 10³ CD8 T lymphocytes specific for the peptide 1073-1081 (CINGVCWTV) (SEQ ID NO:44) obtained from the spleen of HHD mice previously immunized with said peptide together with Poly(I:C) and anti-CD40 as described in Zabaleta et al (Antiviral Res. 2007 April; 74(1):25-35). The DCs and the T lymphocytes were cultured in 96-well ELISPOT plates (Multiscreen HTS; Millipore) and 24 hours later the number of IFN-gamma-producing cells was measured using a commercial BD-Pharmingen kit according to the manufacturer's instructions. The spots were counted with an ELISPOT counter (CTL; Aalen, Germany). The results are shown in FIG. 5.

### Example 6

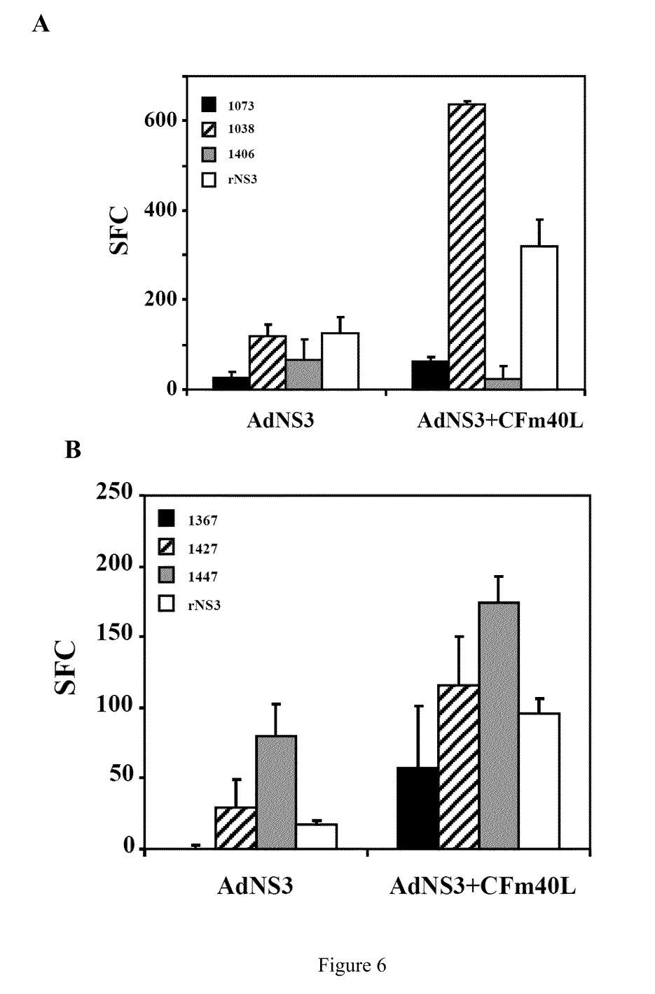

The immunization with DCs transduced with AdNS3 together with CFm40L induces more potent responses than with DCs and AdNS3 alone. (A) DCs from HHD mice were prepared and transduced with AdNS3 at a moi of 30 in the presence or absence of CFm40L and were collected 24 hours later. 2×10⁵ DCswere injected subcutaneously into the base of the tail of HHD mice (n=3 per group) and the animals were sacrificed one week later. An ELISPOT assay was used to measure the frequency of IFN-y-producing cells using a commercial BD-Pharmingen kit according to the manufacturer's instructions. The splenocytes (5×10⁵/well) were cultured in ELISPOT plates. After washing with PBS and blocking with CM with 10% horse serum, the cells were cultured in triplicate in the absence or presence of the synthetic peptides corresponding to HCV NS3 CD8 epitopes 1038-1047 (GLLGCIITSL) (SEQ ID NO:45), 1073-1081 (CINGVCWTV) (SEQ ID NO:44), 1406-1415 (KLVGLGINAV) (SEQ ID NO:46) (all of them at 10 μM) or recombinant NS3 protein (Mikrogen, Neuried, Germany) (1 μg/ml) in HL-1 medium. The number of IFN-γ-producing cells was measured one day later according to the manufacturer's instructions. The results show the points obtained in the presence of antigen except for those obtained in the wells cultured without antigen. (B) A similar experiment was conducted in the C57B16 mouse strain, but in this case the NS3 peptides 1367, 1427 and 1447, described in Zabaleta et al, (Mol Ther. 2008, 16:210-7), were used as antigens. The results are shown in FIG. 6.

### Example 7

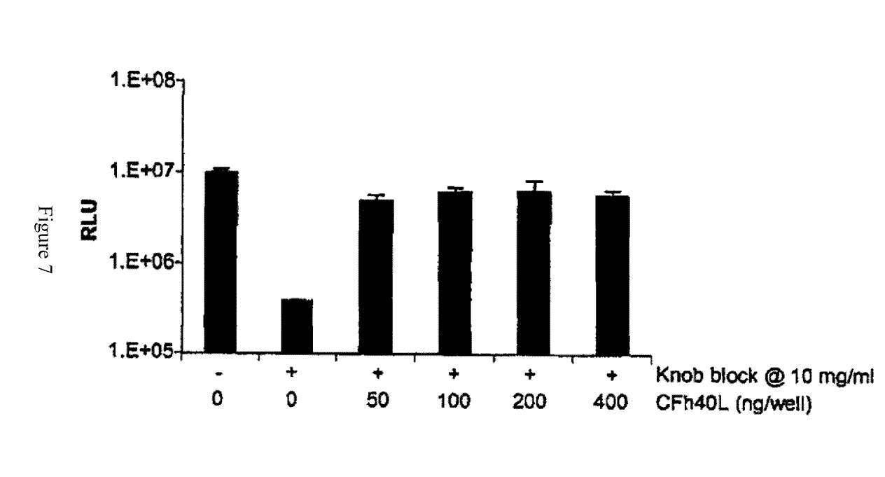

CFh40L, enhances Ad transduction of human CD40-expressing cells. 293 cells stably expressing human CD40 were transduced with Ad encoding luciferase at MOI of 500 v.p./cell. To block the natural Ad receptor CAR the cells were treated with recombinant Ad5 knob at 10 mg/ml. Ad was complexed with CFh40L adapter at indicated concentration and transferred to the cells, in triplicates. Two days later the cells were lysed and luciferase activity measured as relative light units. The results are shown in FIG. 7.

### Example 8

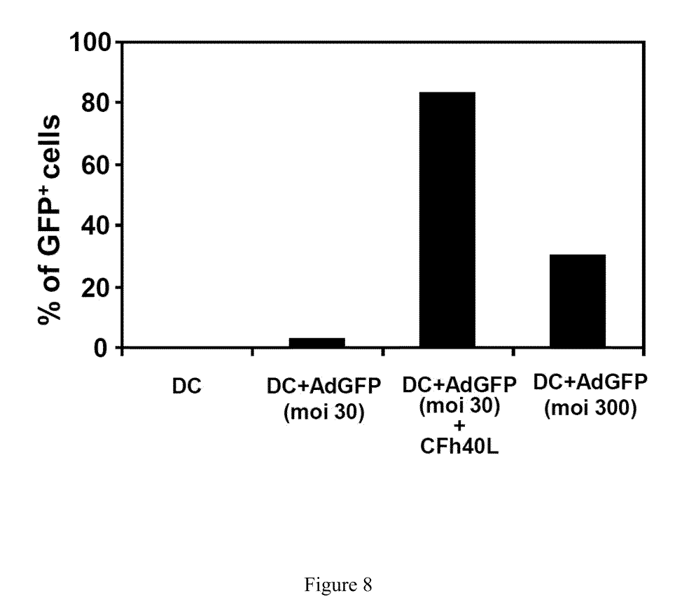

The use of CFh40L increases the efficacy of transduction of DCs with adenoviruses. Human DCs were prepared from monocytes obtained from blood samples from the Banco de Sangre de Navarra (Blood Bank of Navarre). The blood was centrifuged in a Ficoll gradient to purify the mononuclear cells and afterwards the CD14+ monocytes were separated by means of magnetic beads using a Miltenyi kit, according to the manufacturer. After the purification of the monocytes, they were cultured in CM with 1000 U/ml of human GM-CSF and 500 U/ml of human IL-4 (both from Peprotech). After three days of culture, half the medium was changed for fresh medium, also with cytokines and the culture was continued until day 7. The cells were collected, and infected with AdGFP at a moi of 30 in the presence or absence of the adapter CFh40L and at a moi of 300 without adapter CFh40L. After one hour of incubation, CM supplemented with cytokines was added until diluting the cells to a final concentration of 10⁶ cells/ml. The cells were collected 24 hours later and thoroughly washed in order to completely remove adenoviral particles. After the washing, the cells obtained were used for their analysis by flow cytometry. The results are represented in FIG. 8 as the percentage of transduced cells (GFP+) for each of the amounts of virus used.

### Example 9

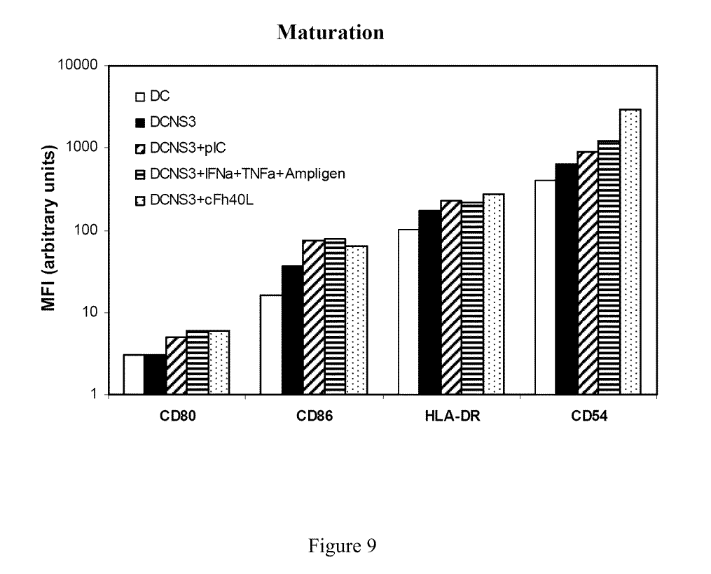

The transduction of human DCs with AdNS3 in the presence of CFh40L induces their in vitro maturation: expression of surface markers Human DCs were prepared from monocytes as described in example 8. The cells were collected and infected with AdNS3 at a moi of 30 in the presence or absence of the adapter CFh40L. In some cases, in a comparative manner, another maturation stimulus such as poly(I:C) (Amersham Biosciences, Piscataway, N.J.) (20 μg/ml) or a cocktail containing TNF-α (Beromune, Boehringer Ingelheim, 200 ng/ml)+Ampligen (HEMISPHERx Biopharma, Philadelphia, USA) (25 μg/ml)+IFN-α (Intron-A, Schering Plough, 1000 U/ml) was added after the infection. After 24 hours, the cells were collected and the expression of the markers CD54, CD80, CD86 and HLA-DR was analyzed by means of flow cytometry, using antibodies against these molecules (all of them from BD-Pharmingen) as explained in example 2. The results are shown in FIG. 9 as the values of each of the markers as the Mean Fluorescence Index (MFI).

### Example 10

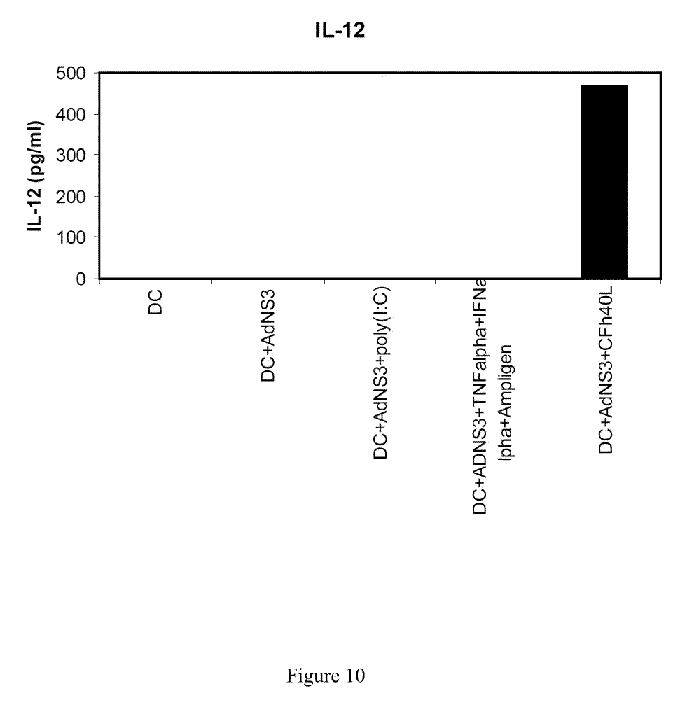

The transduction of human DCs with AdNS3 in the presence of CFh40L induces their in vitro maturation: production of IL-12. Human DCs were prepared from monocytes as indicated in FIG. 7 and treated with AdNS3, AdNS3+TNF-α+Ampligen (HEMISPHERx Biopharma, Philadelphia, USA)+IFNα or AdNS3+CFh40L. After 24 hours, the supernatants were collected and the amount of IL-12 was measured by means of ELISA (BD-Pharmingen). The results are shown in FIG. 10.

### Example 11

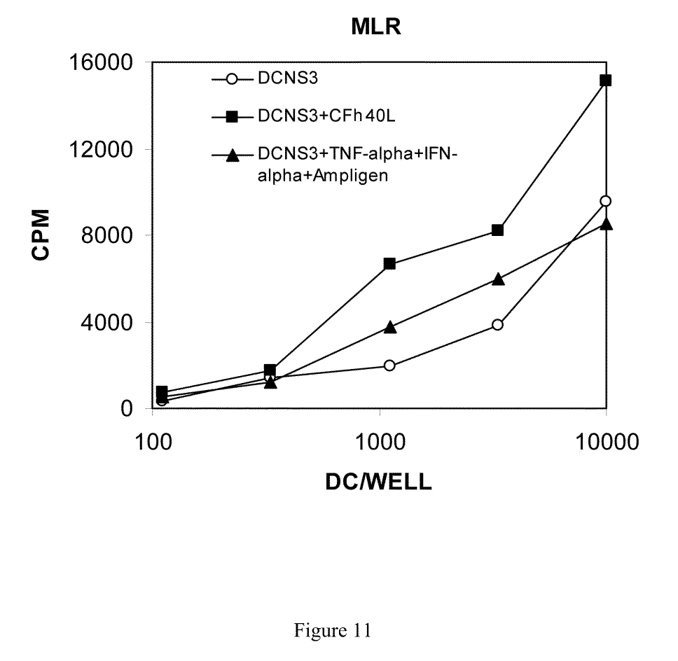

The transduction of human DCs with AdNS3 in the presence of CFh40L induces their in vitro maturation: stimulation of allogeneic T cells. Different amounts of human DCs subjected to the treatments with AdNS3, AdNS3+TNF-α+Ampligen (HEMISPHERx Biopharma, Philadelphia, USA)+IFN-α or AdNS3+CFh40L were cultured together with 10⁵ non-adherent mononuclear cells obtained from another individual. Four days later, 0.5 μCi of [³H] thymidine were added per well and left for another 18 hours. After this time, the samples were collected in Unifilter plates (PerkinElmer, Belgium). Once dried, scintillation fluid was added to the plate and the incorporated thymidine was measured in a scintillation counter as described in example 5. The results are shown in FIG. 11.

### Example 12

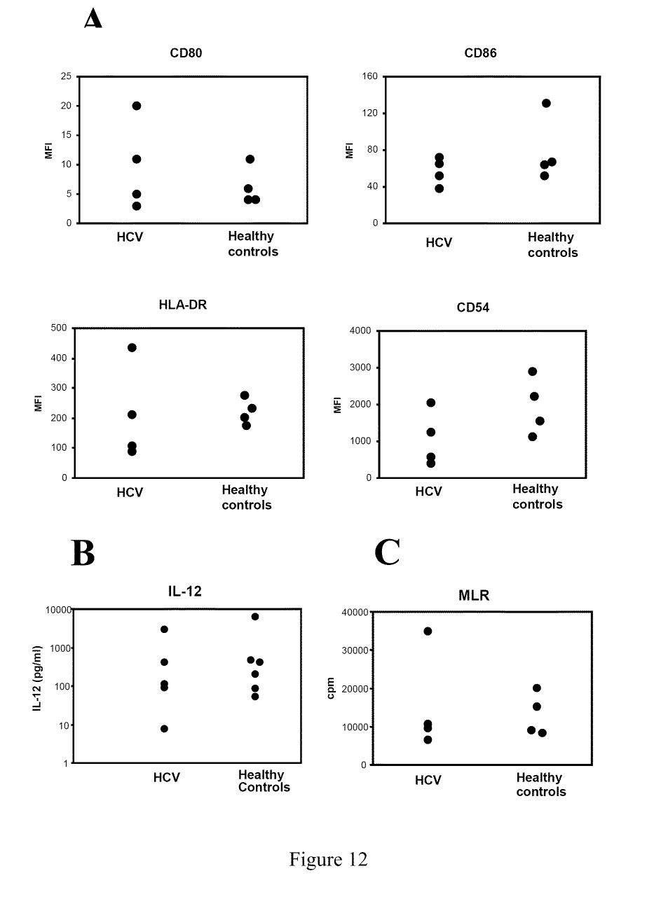

The transduction with AdNS3 of DCs derived from monocytes obtained from patients with chronic hepatitis C virus infection, in the presence of CFh40L, induces a cellular activation similar to that found in DCs obtained from healthy HCV-seronegative individuals. DCs were prepared from monocytes obtained from patients with chronic hepatitis C or from healthy individuals. After 7 days, they were transduced with AdNS3 in the presence of CFh40L and 24 hours later, the expression of surface markers by flow cytometry (A), the production of IL-12 of the culture supernatants (B) and their capacity to stimulate allogeneic T lymphocytes (C) were analyzed. The results are shown in FIG. 12.

1-33. (canceled)

34. A method of obtaining an antigen-loaded CD40-positive antigen-presenting cell, comprising the steps of
(i) contacting a CD40-positive antigen-presenting cell isolated from a patient infected with HCV with a polypeptide comprising
(i) a domain of coxsackievirus and adenovirus receptor (CAR) capable of binding to an adenoviral fiber protein or a functional variant thereof,
(ii) a trimerization motif and
(iii) a human CD40 ligand
and an adenovirus encoding an antigen, wherein said contacting can be carried out by separately adding the polypeptide and the adenovirus or by adding a preformed polypeptide-adenovirus complex,
(ii) maintaining the mixture obtained in step (i) under conditions adequate for the formation of a ternary complex between said polypeptide, said adenovirus and said cell and
(iii) maintaining the cells under conditions adequate for internalization, processing and presentation of one or more peptides derived from the antigen.

35. The method according to claim 34, wherein the domain of CAR comprises an ecto-domain of CAR, preferably amino acids 1-236 of SEQ ID NO:1.

36. The method according to claim 34 wherein the human CD40 ligand comprises amino acids 118 to 231 of SEQ ID NO:6.

37. The method according to claim 34 wherein the trimerization motif comprises a fragment of a bacteriophage T4 fibritin protein.

38. The method according to claim 37, wherein the fragment of the bacteriophage T4 fibritin protein comprises SEQ ID NO:4.

39. The method according to claim 34 further comprising a tag.

40. The method according to claim 39, wherein the tag is a polyhistidine tag, preferably an hexahistidine tag.

41. The method according to claim 34 further comprising a peptide linker at the C-terminus of the ecto-domain of CAR.

42. The method according to claim 41, wherein the peptide linker comprises SEQ ID NO:11.

43. The method according to claim 34 wherein the polypeptide comprises in order from the amino terminal end an ecto-domain of CAR, the trimerization motif, and the human CD40 ligand.

44. The method according to claim 34 further comprising isolating the antigen-loaded, antigen-presenting cell.

45. The method according to claim 44 wherein the CD40-positive antigen-presenting cell is a dendritic cell.

46. A method as defined in claim 34 wherein the antigen is an HCV antigen.

47. A method as defined in claim 46 wherein the HCV antigen is an NS3 protease or an antigenic fragment thereof

48. An antigen-loaded CD40-positive antigen-presenting cell obtained by the method of claim 34.

49. An antigen-loaded CD40-positive antigen-presenting cell as defined in claim 48 for eliciting an immune response in a subject.

50. A method of eliciting an immune response in a subject comprising administration to a subject of the antigen presenting cell of claim 48.

51. A method as defined in claim 50 wherein the antigen presenting cell is a dendritic cell autologous to the subject to be treated.


SEQUENCE LISTING (46 sequences)

SEQ ID NO: 1 - protein, 365 aa, Homo sapiens
MALLLCFVLLCGVVDFARSLSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAEMTSSVISVKNASSEYSGTYSCTVRNRVGSDQCLLRLNVVPPSNKAGLIAGAIIGTLLALALIGLIIFCCRKKRREEKYEKEVHHDIREDVPPPKSRTSTARSYIGSNHSSLGSMSPSNMEGYSKTQYNQVPSEDFERTPQSPTLPPAKVAAPNLSRMGAIPVMIPAQSKDGSIV

SEQ ID NO: 2 - protein, 365 aa, Rattus norvegicus
MALLLCFVLLCGVADFTSSLSITTPEQRIEKAKGETAYLPCKFTLEPEDQGPLDIEWLISPSDNQKVDQVIILYSGDKIYDNYYPDLKGRVHFTSNDVKSGDASINVTNLQLSDIGTYQCKVKKAPGVANRKFLLTVLVKPSGTRCFVDGSGEIGNDFKLKCEPKEGSLPLQYEWQKLSDSQKMPTPWLAEMTSPVISVKNASSEYSGTYSCTVQNRVGSDQCMLRLDVVPPSNRAGTIAGAVIGTLLALVLIGAILFCCHKKRREEKYEKEVHHDIREDVPPPKSRTSTARSYIGSNHSSLGSMSPSNMEGYSKTQYNQVPSEDFERAPQSPTLAPAKVAAPNLSRMGAVPVMIPAQSKDGSIV

SEQ ID NO: 3 - protein, 365 aa, Mus musculus
MARLLCFVLLCGIADFTSGLSITTPEQRIEKAKGETAYLPCKFTLSPEDQGPLDIEWLISPSDNQIVDQVIILYSGDKIYDNYYPDLKGRVHFTSNDVKSGDASINVTNLQLSDIGTYQCKVKKAPGVANKKFLLTVLVKPSGTRCFVDGSEEIGNDFKLKCEPKEGSLPLQFEWQKLSDSQTMPTPWLAEMTSPVISVKNASSEYSGTYSCTVQNRVGSDQCMLRLDVVPPSNRAGTIAGAVIGTLLALVLIGAILFCCHRKRREEKYEKEVHHDIREDVPPPKSRTSTARSYIGSNHSSLGSMSPSNMEGYSKTQYNQVPSEDFERAPQSPTLAPAKVAAPNLSRMGAVPVMIPAQSKDGSIV

SEQ ID NO: 4 - protein, 26 aa, T4 phage
GYIPEAPRDGQAYVRKDGEWVLLSTF

SEQ ID NO: 5 - protein, 36 aa, Homo sapiens
PDVASLRQQVEALQGQVQHLQAAFSQYKKVELFPNG

SEQ ID NO: 6 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 7 - protein, 14 aa, Artificial
  Linker
SGGTSGSTSGTGST

SEQ ID NO: 8 - protein, 15 aa, Artificial
  Linker
AGSSTGSSTGPGSTT

SEQ ID NO: 9 - protein, 7 aa, Artificial
  Linker
GGSGGAP

SEQ ID NO: 10 - protein, 8 aa, Artificial
  Linker
GGGVEGGG

SEQ ID NO: 11 - protein, 5 aa, Artificial
  Linker
GGPGS

SEQ ID NO: 12 - protein, 635 aa, Hepatitis C virus
APITAYSQQTRGLLGCIITSLTGRDKNQVDGEVQVLSTATQSFLATCVNGVCWTVYHGAGSKTLAGPKGPITQMYTNVDQDLVGWPAPPGARSMTPCTCGSSDLYLVTRHADVVPVRRRGDSRGSLLSPRPISYLKGSSGGPLLCPSGHVVGIFRAAVCTRGVAKAVDFIPVESMETTMRSPVFTDNSSPPAVPQTFQVAHLHAPTGSGKSTKVPAAYAAQGYKVLVLNPSVAATLGFGAYMSKAHGIEPNIRTGVRTITTGGPITYSTYCKFLADGGCSGGAYDIIICDECHSTDSTTILGIGTVLDQAETAGARLVVLATATPPGSITVPHPNIEEVALSNTGEIPFYGKAIPIEAIKGGRHLIFCHSKKKCDELAAKLTGLGLNAVAYYRGLDVSVIPTSGDVVVVATDALMTGFTGDFDSVIDCNTCVTQTVDFSLDPTFTIETTTLPQDAVSRAQRRGRTGRGRSGIYRFVTPGERPSGMFDSSVLCECYDAGCAWYELTPAETSVRLRAYLNTPGLPVCQDHLEFWESVFTGLTHIDAHFLSQTKQAGDNLPYLVAYQATVCARAQAPPPSWDQMWKCLIRLKPTLHGPTPLLYRLGAVQNEVTLTHPITKYIMACMSADLEVVTSTWV

SEQ ID NO: 13 - protein, 10 aa, Hepatitis C virus
TGAPVTYSTY

SEQ ID NO: 14 - protein, 10 aa, Hepatitis C virus
VLSTATQSFL

SEQ ID NO: 15 - protein, 9 aa, Hepatitis C virus
KGSSGGPLL

SEQ ID NO: 16 - protein, 9 aa, Hepatitis C virus
LLCPSGHVV

SEQ ID NO: 17 - protein, 10 aa, Hepatitis C virus
FIPVESMETT

SEQ ID NO: 18 - protein, 10 aa, Hepatitis C virus
SSPPAVPQTF

SEQ ID NO: 19 - protein, 9 aa, Hepatitis C virus
AYMSKAHGI

SEQ ID NO: 20 - protein, 11 aa, Hepatitis C virus
RTGVRTITTTG

SEQ ID NO: 21 - protein, 9 aa, Hepatitis C virus
RTITTGGPI

SEQ ID NO: 22 - protein, 9 aa, Hepatitis C virus
TYSTYCKFL

SEQ ID NO: 23 - protein, 9 aa, Hepatitis C virus
TILGIGTVL

SEQ ID NO: 24 - protein, 10 aa, Hepatitis C virus
IGTVLDQAET

SEQ ID NO: 25 - protein, 9 aa, Hepatitis C virus
VALSNTGEI

SEQ ID NO: 26 - protein, 10 aa, Hepatitis C virus
AIPIEAIKGG

SEQ ID NO: 27 - protein, 10 aa, Hepatitis C virus
KCDELAAKLT

SEQ ID NO: 28 - protein, 10 aa, Hepatitis C virus
VVVVATDALM

SEQ ID NO: 29 - protein, 9 aa, Hepatitis C virus
TFTIETTTL

SEQ ID NO: 30 - protein, 10 aa, Hepatitis C virus
VDFSLDPTFT

SEQ ID NO: 31 - protein, 10 aa, Hepatitis C virus
DAVSRAQRRG

SEQ ID NO: 32 - protein, 9 aa, Hepatitis C virus
AYLNTPGLP

SEQ ID NO: 33 - protein, 9 aa, Hepatitis C virus
SVFTGLTHI

SEQ ID NO: 34 - protein, 9 aa, Hepatitis C virus
LTHIDAHFL

SEQ ID NO: 35 - protein, 9 aa, Hepatitis C virus
CLIRLKPTL

SEQ ID NO: 36 - protein, 10 aa, Hepatitis C virus
GPTPLLYRLG

SEQ ID NO: 37 - protein, 9 aa, Hepatitis C virus
LTHPITKYI

SEQ ID NO: 38 - DNA, 20 bp, Artificial
  Forward primer DLL4
gtgggtaagatttggcgaac

SEQ ID NO: 39 - DNA, 20 bp, Artificial
  DLL4 reverse
gtgggggatacattcattgc

SEQ ID NO: 40 - DNA, 20 bp, Artificial
  Jagged 1 forward
tatctgtccacctggctatg

SEQ ID NO: 41 - DNA, 20 bp, Artificial
  Jagged 1 reverse
agtcactggcacgattgtag

SEQ ID NO: 42 - DNA, 20 bp, Artificial
  Jagged 2 forward
tcgtcgtcattccctttcag

SEQ ID NO: 43 - DNA, 20 bp, Artificial
  Jagged 2 reverse
gtggcactgtagtagttctc

SEQ ID NO: 44 - protein, 9 aa, Artificial
  HCV NS3 peptide
CINGVCWTV

SEQ ID NO: 45 - protein, 10 aa, Artificial
  HCV NS3 CD8 epitope
GLLGCIITSL

SEQ ID NO: 46 - protein, 10 aa, Artificial
  HCV NS3 CD8 epitope
KLVGLGINAV